(19) United States
(12) Patent Application Publication
(10) Pub. No.: US 2026/0191528 A1
(43) Pub. Date: Jul. 9, 2026

(54) CARTRIDGE, END TOOL, AND SURGICAL INSTRUMENT

(71) Applicant: LIVSMED INC., Seongnam-si (KR)

(72) Inventor: Woo Jung CHOI, Siheung-si (KR)

(21) Appl. No.: 19/354,389

(22) Filed: Oct. 9, 2025

(30) Foreign Application Priority Data
Jan. 8, 2025 (KR) 10-2025-0003060

(51) Int. Cl. A61B 17/072; A61B 17/00

(52) U.S. Cl. CPC A61B 17/072; A61B 2017/00477; A61B 2017/07271; A61B 2017/07278

(57) ABSTRACT

A cartridge of a surgical instrument, which is applied to an end tool of the surgical instrument, includes a housing, a cover covering one surface of the housing and having a slit defined along a first direction, which is a longitudinal direction of the housing, a plurality of staples disposed inside the housing, and a moving member disposed inside the housing, movable along the slit relative to the housing, and having a deformable region that is configured to be deformable according to an external force.

### CROSS-REFERENCE TO RELATED APPLICATION

This application is based on and claims priority under 35 USC §119 to Korean Patent Application No. 10-2025-0003060, filed on January 08, 2025, in the Korean Intellectual Property Office, the disclosure of which is incorporated by reference herein in its entirety.

### BACKGROUND

### 1. Field

The present disclosure relates to a cartridge, an end tool, and a surgical instrument.

### 2. Description of the Related Art

Medically, surgery refers to the treatment of diseases by cutting, slitting, or manipulating the skin, mucous membranes, or other tissues using medical devices In particular, open surgery, which cuts and opens the skin of a surgical site and cures, shapes, or removes an organ therein, may cause bleeding, side effects, patient pain, scars, or the like. Accordingly, recently, surgery performed by inserting only a medical device, for example, laparoscopic surgical instrument, microsurgical microscope, and the like by forming a predetermined hole in the skin or surgery using a robot has been spotlighted as an alternative.

A surgical instrument is a tool equipped with an end tool provided on one end of a shaft that passes through a hole drilled in the skin, and is manipulated by a medical doctor by hand using a predetermined driving part or by a robot arm to perform surgery at the surgical site. The end tool provided on the surgical instrument performs a rotational motion, a gripping motion, a cutting motion, or the like through a predetermined structure.

The background art described above is technical information retained by the present inventors in order to derive the present disclosure or obtained by the present inventors in the process of deriving the present disclosure, and thus is not necessarily known art disclosed to the general public before the filing of the present application.

### SUMMARY

The present disclosure is directed to providing a cartridge, an end tool, and a surgical instrument that enable a formed height of a staple to be flexibly adjusted within a predetermined range during laparoscopic surgery or various other surgical procedures.

According to an aspect of the present disclosure, a cartridge applied to an end tool of a surgical instrument may include a housing, a cover that covers one surface of the housing and has a slit defined along a first direction, which is a longitudinal direction of the housing, a plurality of staples positioned inside the housing, and a moving member positioned inside the housing, movable along the slit relative to the housing, and having a region that is deformable according to an external force.

In another embodiment of the present disclosure, the region of the moving member may have a lower rigidity than other regions.

In the other embodiment of the present disclosure, the moving member may include a body part positioned in the slit, and a wedge part positioned on each of opposite sides of the body part and configured to push the plurality of staples upward.

In the other embodiment of the present disclosure, the region may be positioned on the wedge part, and the wedge part may include a contact surface configured to contact the plurality of staples and a bottom surface opposite to the contact surface, wherein the contact surface may be deformable by the external force.

In the other embodiment of the present disclosure, the wedge part of the moving member may include a wedge hole, which is positioned at a first predetermined height of the region and extends in the first direction.

In the other embodiment of the present disclosure, the wedge hole may be positioned closer to the contact surface than to the bottom surface.

In the other embodiment of the present disclosure, the wedge part may include first end positioned toward a distal end of the cartridge and second end positioned toward a proximal end of the cartridge, and the wedge hole may be positioned closer to the second than to the first end.

In the other embodiment of the present disclosure, the contact surface of the moving member may have a certain inclined surface with respect to the first direction, and at least a portion of the wedge hole may be formed in a direction parallel to the inclined surface.

In the other embodiment of the present disclosure, the wedge part of the moving member may include a wedge groove, which is positioned at a second predetermined height of the region and open in a direction toward a proximal end of the cartridge.

In the other embodiment of the present disclosure, the region may be positioned on the wedge part, and the wedge part may include a contact surface configured to contact the plurality of staples and a bottom surface opposite to the contact surface, wherein the bottom surface may be deformable by the external force.

In the other embodiment of the present disclosure, the wedge part may have a protrusion that protrudes from the bottom surface toward the housing and extends in the first direction.

In the other embodiment of the present disclosure, the wedge part may further include a hollow positioned adjacent to the protrusion and formed in the first direction.

According to another aspect of the present disclosure, an end tool may include a first jaw configured to accommodate at least one region of an operation member that is movable in a first direction, a second jaw facing the first jaw and configured to be rotatable around the first jaw, and a cartridge including a cover positioned in the first jaw and having a slit defined along the first direction, and a plurality of staples configured to be withdrawn toward the second jaw in response to a movement of the operation member, wherein a height of each of the plurality of staples is adjusted by a force applied when the plurality of staples are withdrawn.

In the other embodiment of the present disclosure, the cartridge may further include a moving member configured to push the plurality of staples upward in response to the movement of the operation member, wherein the moving member may have a region that is deformable according to an external force.

In the other embodiment of the present disclosure, the moving member may include a body part positioned in the slit, and a wedge part positioned on each of opposite sides of the body part and configured to push the plurality of staples upward.

In the other embodiment of the present disclosure, the region may be positioned on the wedge part, and the wedge part may include a contact surface configured to contact the plurality of staples and a bottom surface opposite to the contact surface, wherein the contact surface may be deformable by the external force.

In the other embodiment of the present disclosure, the wedge part of the moving member may include a wedge hole, which is positioned at a first predetermined height of the region and extends in the first direction.

In the other embodiment of the present disclosure, the wedge hole may be positioned closer to the contact surface than to the bottom surface.

In the other embodiment of the present disclosure, the wedge part of the moving member may include a wedge groove, which is positioned at a second predetermined height of the region and open in a direction toward a proximal end of the end tool.

In the other embodiment of the present disclosure, the region may be positioned on the wedge part, and the wedge part may include a contact surface configured to contact the plurality of staples and a bottom surface opposite to the contact surface, wherein the bottom surface may be deformable by the external force.

In the other embodiment of the present disclosure, the wedge part may have a protrusion that protrudes from the bottom surface toward the first jaw and extends in the first direction.

According to another aspect of the present disclosure, a surgical instrument may include an end tool including a first jaw configured to accommodate at least one region of an operation member that is movable in a first direction, a second jaw facing the first jaw and configured to be rotatable around the first jaw, and a cartridge including a cover disposed in the first jaw and having a slit defined along the first direction, and a plurality of staples configured to be withdrawn toward the second jaw in response to a movement of the operation member, in which a height of each of the plurality of staples is adjusted by a force applied when the plurality of staples are withdrawn, a manipulation part configured to control an operation of the end tool, and a connection part configured to connect the manipulation part to the end tool.

In the present embodiment, the end tool may be configured to perform a yaw rotation around one shaft and a pitch rotation around another shaft different from the one shaft.

Other aspects, features, and advantages other than those described above will become apparent from the following drawings, claims, and detailed description of the disclosure.

### BRIEF DESCRIPTION OF THE DRAWINGS

The above and other aspects, features, and advantages of certain embodiments of the disclosure will be more apparent from the following description taken in conjunction with the accompanying drawings.

FIG. 1 is a perspective view illustrating a surgical instrument to which an end tool according to an embodiment of the present disclosure is applied.

FIG. 2 is a perspective view illustrating a reload assembly to which the end tool according to an embodiment of the present disclosure is applied.

FIG. 3 is a perspective view illustrating the end tool according to an embodiment of the present disclosure.

FIG. 4 is a perspective view of the end tool of FIG. 3 with a cartridge removed, viewed from another direction.

FIG. 5 is a perspective view of the end tool of FIG. 3 with a second jaw removed.

FIG. 6 is a schematic perspective view for describing an operation member, a moving member, fixed pulleys, and advancing wires of the end tool of FIG. 3.

FIGS. 7A to 7C are schematic views for describing operations of the operation member of the end tool of FIG. 6.

FIG. 8 is a perspective view of the cartridge of FIG. 3.

FIG. 9 is an exploded perspective view of the cartridge of FIG. 8.

FIG. 10 is a perspective view of the cartridge of FIG. 8 with a portion of a case of the cartridge removed.

FIG. 11 is an exploded perspective view illustrating the end tool and the cartridge of FIG. 3.

FIGS. 12A to 13 are cross-sectional views illustrating an overall staple operation of the end tool of the surgical instrument of FIG. 1.

FIG. 14 is a perspective view of the moving member of the cartridge of FIG. 9.

FIG. 15 is a side view of the moving member of FIG. 14.

FIG. 16 is a perspective view of the moving member of FIG. 14, viewed from another direction.

FIG. 17 is a cross-sectional view illustrating an overall staple operation of the moving member of FIG. 14.

FIG. 18 is a perspective view of a moving member according to another embodiment of the present disclosure.

FIG. 19 is a side view of the moving member of FIG. 18.

FIG. 20 is a perspective view of the moving member of FIG. 18, viewed from another direction.

FIG. 21 is a perspective view of a moving member according to the other embodiment of the present disclosure.

FIG. 22 is a side view of the moving member of FIG. 21.

FIG. 23 is a perspective view of the moving member of FIG. 21, viewed from another direction.

### DETAILED DESCRIPTION

Hereinafter, the following embodiments will be described in detail with reference to the accompanying drawings. When describing with reference to the drawings, identical or corresponding components will be assigned the same reference numerals and duplicate descriptions thereof will be omitted.

Since various transformations can be made to these embodiments, specific embodiments will be illustrated in the drawings and described in detail in the detailed description. The effects and features of the present embodiments and the accompanying methods thereof will become apparent from the following description of the contents, taken in conjunction with the accompanying drawings. However, the present embodiments are not limited to the embodiments disclosed below, but may be implemented in various forms.

In describing the present disclosure, a detailed description of known related arts will be omitted when it is determined that the gist of the present disclosure may be unnecessarily obscured.

In the following embodiments, singular forms are intended to include plural forms as well, unless the context clearly indicates otherwise. Although terms such as “first,” “second,” and the like may be used to describe various components, such components should not be limited to the above terms The terms are only used to distinguish one component from another.

In the following embodiments, terms such as “include” or “have” means that the features or components described in the specification are present, and the possibility that one or more other features or components will be added is not excluded in advance.

In the following embodiments, when a unit, region, or component is referred to as being formed on another unit, region, or component, it can be directly formed on the other unit, region, or component. That is, for example, intervening units, regions, or components may be present.

In the following embodiments, terms such as “connecting” or “coupling” two members do not necessarily mean a direct and/or fixed connection or coupling of the two members, unless the context clearly indicates otherwise, and do not preclude another members from being interposed between the two members.

Sizes of components in the drawings may be exaggerated or reduced for convenience of description. For example, since the size and thickness of each component shown in the drawings are arbitrarily illustrated for convenience of description, the following embodiments are not necessarily limited thereto.

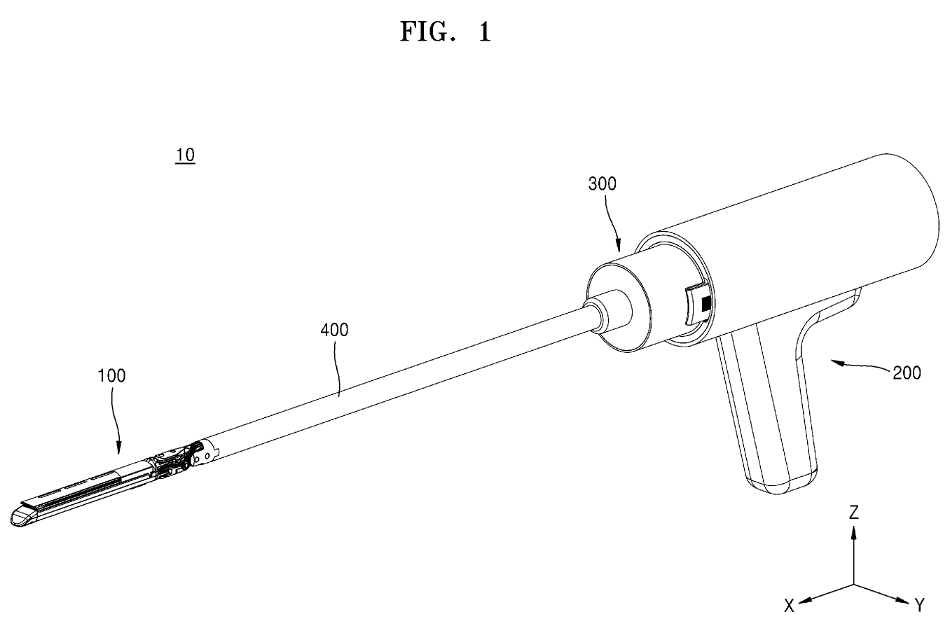

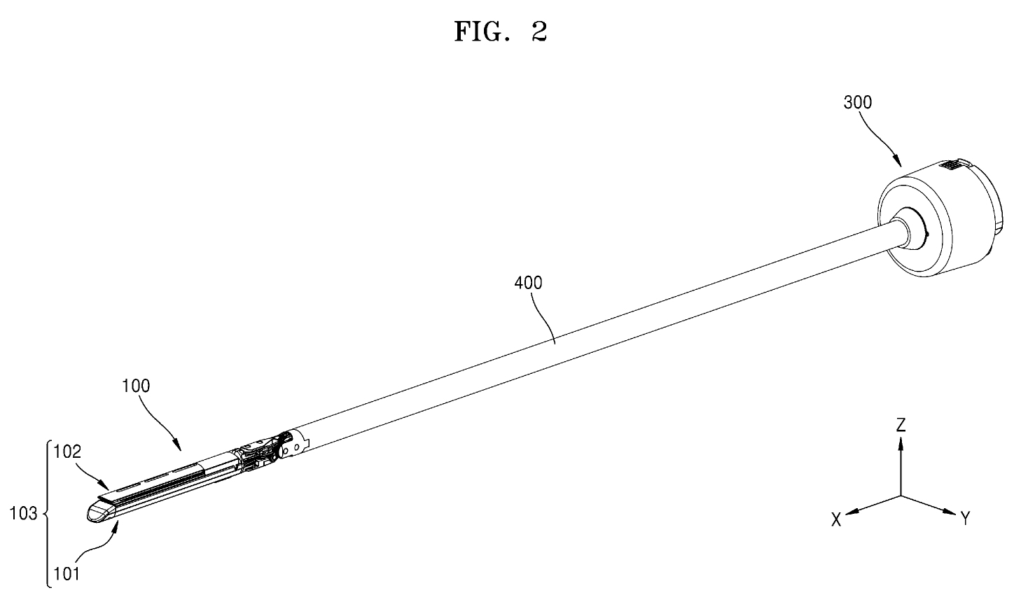

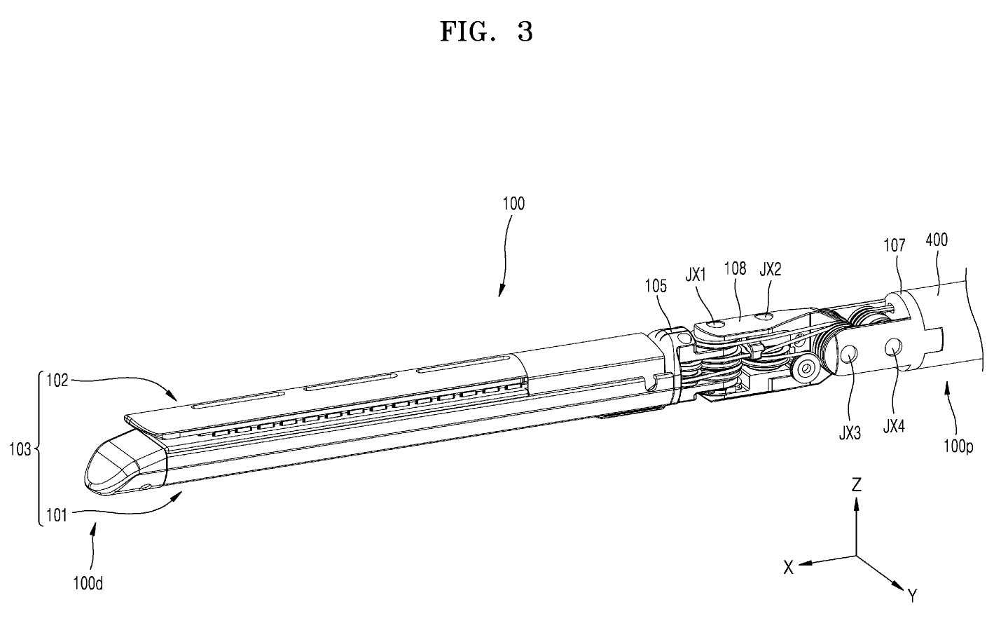

FIG. 1 is a perspective view illustrating a surgical instrument to which an end tool according to an embodiment of the present disclosure is applied, and FIG. 2 is a perspective view illustrating a reload assembly to which the end tool according to an embodiment of the present disclosure is applied. FIG. 3 is a perspective view illustrating the end tool according to an embodiment of the present disclosure.

In describing the present disclosure, the portion closer to a user side, i.e., the portion closer to a manipulation part 200, will be referred to as a proximal end, and the portion farther from the user side, that is, the portion closer to an end portion of an end tool 100, will be referred to as a distal end.

For example, in the end tool 100, the portion closer to a connection part (or shaft) 400 will be defined as a proximal end 100p, and the portion farther from the connection part (or shaft) 400, that is, the portion closer to an end portion of the end tool 100 will be defined as a distal end 100d and described accordingly.

For example, for convenience of description, even in a cartridge 500 mounted on the end tool 100, the portion of the cartridge 500 closer to the distal end of the end tool 100 will be defined as the proximal end 100p, and the portion closer to the manipulation part 200 will be defined as the distal end 100d and described accordingly.

For example, a longitudinal direction of the end tool 100, a moving direction of an operation member 140, an X-axis direction, and a longitudinal direction of the cartridge 500 will be defined as a first direction and described accordingly.

The end tool 100 according to an embodiment of the present disclosure may be connected to one end portion of the connection part (or shaft) of the surgical instrument and inserted into a surgical site to perform motions required for surgery.

First, the surgical instrument and the reload assembly to which the end tool 100 according to an embodiment of the present disclosure is applicable will be described.

Referring to FIGS. 1 to 3, the surgical instrument according to the present embodiment may include the end tool 100, the manipulation part 200, the connection part 400, and a power transmission part 300.

For example, as illustrated in FIG. 2, a configuration including the end tool 100, the connection part (or shaft) 400, and the power transmission part 300 will be distinguished and referred to as the reload assembly.

Here, the connection part 400 is formed in the shape of a hollow shaft, and one or more wires and electric wires may be accommodated therein. For convenience of description of the present embodiment, the shaft will be referred to and described as the connection part 400. The manipulation part 200 is coupled to one end portion of the connection part 400, the end tool 100 is coupled to another end portion thereof, and the connection part 400 may serve to connect the manipulation part 200 to the end tool 100. As an example, the connection part 400 may include a straight part, and although not shown in the drawings, the connection part 400 may include one or more curved parts to increase ease of use and control the arrangement of components for manipulation.

The power transmission part 300 may be formed on one end portion of the connection part 400 and may serve to transmit power generated from a power generation part to be described later to the end tool 100. For example, the power transmission part 300 may be positioned between the end tool 100 and the manipulation part 200. As will be described later, when a user such as a medical doctor manipulates the manipulation part 200, the power generation part generates power to control the end tool 100, and the generated power may be transmitted to the end tool 100 through the power transmission part 300. The power transmission part 300 may include a plurality of wires, a plurality of pulleys, a plurality of links, a plurality of joints, a plurality of gears, and the like.

A user may operate the end tool 100 by manipulating the manipulation part 200. For example, the manipulation part 200 is a configuration for the user to input signals to control the motions of the end tool 100. That is, the manipulation part 200 may be said to be a configuration that receives signals from the user to control the operation of the end tool 100. Here, the signals for controlling the motions of the end tool 100 may correspond to mechanical manipulations such as pressing a button or switch, or rotating or moving a particular member, and may also be electrical signals generated by such mechanical manipulations, but the present disclosure is not limited thereto.

The manipulation part 200 is provided as an interface to be directly controlled by a medical doctor, for example, provided in a gun shape, a tongs shape, a stick shape, a lever shape, or the like, and when the medical doctor controls the manipulation part 200, the end tool 100, which is connected to the corresponding interface and inserted into the body of a surgical patient, performs a certain motion, thereby performing surgery. Here, the manipulation part 200 is illustrated in FIG. 1 as being formed in a gun shape, but the concept of the present disclosure is not limited thereto, and various types of manipulation parts that can be connected to the end tool 100 and manipulate the end tool 100 may be possible.

The manipulation part 200 may include a housing forming an outer shape of the manipulation part 200. At least a portion of the power generation part configured to generate power to control the end tool 100 may be accommodated inside the housing. Further, a circuit unit for controlling the operation of the power generation part and a slip ring for supplying electrical energy to the power generation part, connecting communication, or transmitting various other signals may be accommodated inside the housing.

A handle may be formed on the manipulation part 200. The handle is a part for a user to grip. Thus, the user can use a surgical instrument 10 according to the present disclosure while gripping the handle of the manipulation part 200.

For example, although not shown in the drawings, a button, a switch, a lever, and the like for controlling various motions of the end tool 100 may be further formed in the manipulation part.

The end tool 100 is formed on another end portion of the connection part 400, and performs necessary motions for surgery by being inserted into a surgical site. As an example of the end tool 100, a pair of jaws 103 for performing a grip motion may be used. However, the concept of the present disclosure is not limited thereto, and various devices for performing surgery may be used as the end tool 100. For example, a configuration such as a cantilever cautery may also be used as the end tool. The end tool 100 is connected to the manipulation part 200 by the power transmission part 300, and receives a driving force of the manipulation part 200 through the power transmission part 300 (e.g., a wire or the like) to perform a motion necessary for surgery, such as gripping, cutting, suturing, or the like.

Hereinafter, the end tool 100 of the surgical instrument of FIG. 3 will be described in more detail.

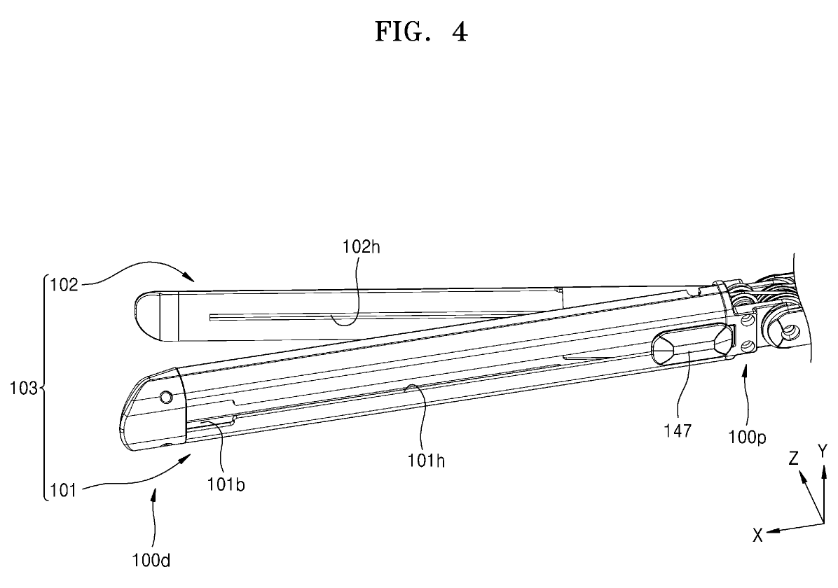

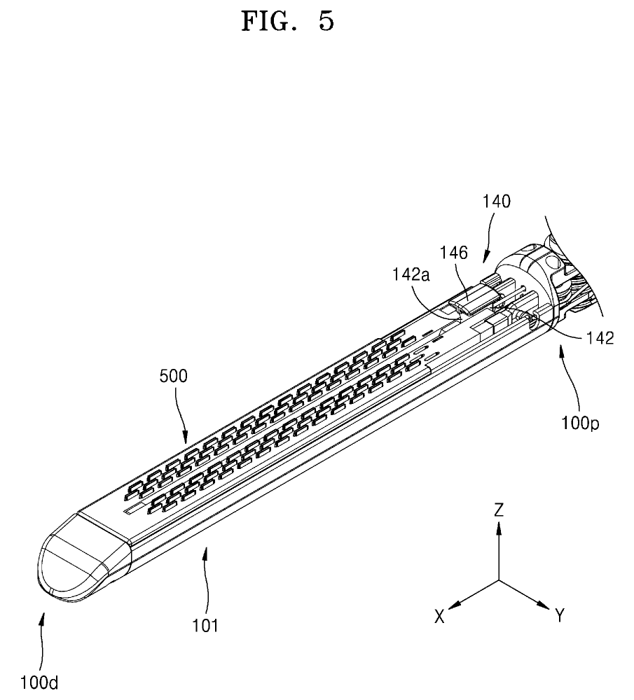

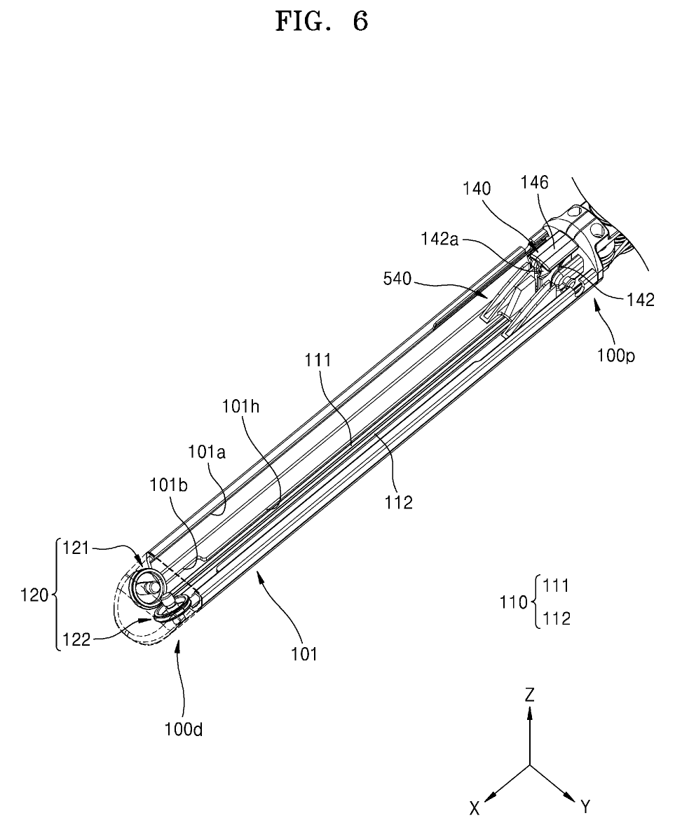

FIG. 4 is a perspective view of the end tool of FIG. 3 with the cartridge removed, viewed from another direction, FIG. 5 is a perspective view of the end tool of FIG. 3 with a second jaw removed, and FIG. 6 is a schematic perspective view for describing the operation member, a moving member, fixed pulleys, and advancing wires of the end tool of FIG. 3.

The end tool 100 may include the jaw 103, a plurality of fixed pulleys 120, and a plurality of advancing wires 110. The plurality of fixed pulleys 120 may include two or more pulleys, for example, a first fixed pulley 121 and a second fixed pulley 122. The plurality of advancing wires 110 include two or more wires, and may include, for example, a first advancing wire 111 and a second advancing wire 112.

The jaw 103 may perform various functions, for example, a grip motion, and may include a pair of jaws, e.g., a first jaw 101 and a second jaw 102 as a specific example. Here, each of the first jaw 101 and the second jaw 102, or a component encompassing the first jaw 101 and the second jaw 102 may be referred to as the jaw 103.

The first jaw 101 and the second jaw 102 may be positioned to face each other, may move closer to or move away from each other, and may be configured to rotationally move around, for example, one shaft.

The cartridge 500 may be positioned to be accommodated in the first jaw 101, and a plurality of staples 530 are positioned inside the cartridge 500. When an operation member 140 receives a force through the advancing wires 110 while the first jaw 101 and the second jaw 102 are close to each other, such as when the first jaw 101 and the second jaw 102 are closed with the body tissue interposed therebetween, the operation member 140 may move toward the distal end 100d of the first jaw 101 while pushing the staples upward, thereby performing stapling. At this point, one or more clamps 146 and 147 of the operation member 140 may protrude to the outside of the first jaw 101 and the second jaw 102, allowing the operation member 140 to move forward while applying a pressure to an outer surface of the first jaw 101 and the second jaw 102, which facilitates the smooth progression of a stapling process. In an optional embodiment, the cartridge 500 may have a housing 520 corresponding to the bottom thereof and may have a form in which the housing 520 is positioned in the first jaw 101.

The operation member 140 may include a body 142, a first clamp 146, and a second clamp 147. For example, the operation member 140 may be used together with the moving member 540. For example, the moving member 540 may be prepared separately from the operation member 140 and then positioned adjacent to the operation member 140 in the first jaw 101. Further, as another example, the operation member 140 and the moving member 540 may be integrally formed as one body. In the present specification, for convenience of description, the operation member 140 and the moving member 540 will be described and illustrated in the drawings with the assumption that the operation member 140 and the moving member 540 are prepared separately.

The moving member 540 may be positioned on at least one side of the body 142 and may have a predetermined inclined surface. That is, the moving member 540 may be inclined by a certain degree with respect to an extension direction of the end tool 100. In other words, the moving member 540 may have a greater height at the proximal end 100p side of the first jaw 101 than the distal end 100d side of the first jaw 101.

The moving member 540 may be configured to be sequentially in contact with withdrawal members 535 or the plurality of staples 530 positioned in the cartridge 500, and may serve to sequentially push the staples 530 upward.

The plurality of fixed pulleys 120 may be disposed in the first jaw 101 to be closer to the front of the cartridge 500, i.e., to the distal end 100d of the first jaw 101, than the cartridge 500, and details thereof will be described later.

Referring to FIG. 3, the end tool 100 of the surgical instrument of the present embodiment may include one or more members, such as joint members, that connect the jaw 103 to the connection part 400. Further, in an optional embodiment, the end tool 100 may include a pitch hub 107 and an end tool hub 108.

The pitch hub 107 and the end tool hub 108 may be positioned to connect the end tool 100 to the straight part of the connection part 400.

As an example, the pitch hub 107 may have a pulley shaft JX3 corresponding thereto, and the pulley shaft JX3 may be a pitch rotation shaft. For example, the end tool 100 may perform a vertical rotational motion around the pulley shaft JX3 based on the drawing. Further, one or more pulleys may be positioned to be adjacent to the pulley shaft JX3.

The pitch hub 107 may be in the form of a bar extending from the center of a surface thereof that corresponds to the connection part 400, i.e., a bar extending from the center of a disk-shaped main region. The pulley shaft JX3 and a pulley shaft JX4 different from the pulley shaft JX3 may further correspond to a region of the bar.

The end tool hub 108 is connected to the pitch hub 107 and the jaw 103. The pitch hub 107 may be axially coupled to the pitch hub 107 with respect to one pulley shaft, i.e., the pulley shaft JX3. The end tool hub 108 may rotationally move around one pulley shaft, i.e., the pulley shaft JX3 while connected to the pitch hub 107. That is, the end tool 100 may perform a pitch motion as the end tool hub 108 rotates around one pulley shaft, i.e., the pulley shaft JX3 relative to the pitch hub 107.

Further, the jaw 103 of the end tool 100 may be axially coupled to the end tool hub 108 with respect to one pulley shaft, i.e., a pulley shaft JX1. The jaw 103 may rotate around one pulley shaft, i.e., the pulley shaft JX1 while connected to the end tool hub 108. That is, the jaw 103 of the end tool 100 may rotate around one pulley shaft, i.e., the pulley shaft JX1 relative to the end tool hub 108, thereby performing a yaw motion.

As a result, the yaw motion of the end tool 100 includes a rotational motion of the jaw 103 around one pulley shaft, i.e., the pulley shaft JX1 relative to the end tool hub 108, and the pitch motion of the end tool 100 includes a rotational motion of the jaw 103 coupled to the end tool hub 108, which occurs as the end tool hub 108 rotates around one pulley shaft, i.e., the pulley shaft JX3 relative to the pitch hub 107.

The operation member 140 of the end tool 100 will be described in more detail.

Referring to, FIGS. 4, 5, and 6, the body 142 may have an elongated columnar shape and, for example, may be in the form of a plate-shaped column. For example, a blade 142a may be formed in one region of the body 142, and an edge sharply formed to cut tissue may be formed in the blade 142a. The tissue positioned between the first jaw 101 and the second jaw 102 may be cut as at least a portion of the edge positioned in the blade 142a of the body 142 is withdrawn to the outside of the first jaw 101 and the cartridge 500.

The first clamp 146 may be formed in one region of the body 142, and the second clamp 147 may be formed in another region different from the one region. For example, the body 142 may be positioned between the first clamp 146 and the second clamp 147.

The first clamp 146 and the second clamp 147 may have a region with a width at least greater than that of the body 142. Accordingly, the first clamp 146 may be inserted into and pass through a guide groove 102h formed in the second jaw 102 in the longitudinal direction to be positioned or brought into contact with an upper surface of the second jaw 102 and, at the same time, the second clamp 147 may be inserted into and pass through a guide groove 101h formed in the first jaw 101 in the longitudinal direction to be positioned or brought into contact with a lower surface of the first jaw 101, so that the first clamp 146 and the second clamp 147 may move. Thus, when the operation member 140 moves, the first clamp 146 and the second clamp 147 may apply forces in directions that bring the second jaw 102 and the first jaw 101 closer to each other.

As a result, when the operation member 140 moves from the proximal end 100p of the first jaw 101 toward the distal end 100d of the first jaw 101, a motion of decreasing a distance between the second jaw 102 and the first jaw 101, i.e., a closing motion of the jaw 103, may be naturally implemented through the first clamp 146 and the second clamp 147.

The first clamp 146 and the second clamp 147 may be located at different positions relative to a forward-facing direction relative to the body 142. For example, the second clamp 147 may be positioned further forward than the first clamp 146, for example, the second clamp 147 may be positioned closer to the distal end 100d of the first jaw 101 than the first clamp 146 when the operation member 140 is positioned in the first jaw 101. Accordingly, the operation member 140 may move forward while the first jaw 101 and second jaw 102 are in the closed state, so that the first jaw 101 and second jaw 102 can be maintained with greater efficiency and stability while performing stapling.

The jaw 103 of the end tool 100 will be described in more detail.

Referring to FIGS. 4 and 6 and the like, the first jaw 101 is formed in an elongated bar shape as a whole, and a rotation shaft may be positioned in the proximal end such that the first jaw 101 is rotationally movable, and such a rotation shaft may correspond to the rotation shaft formed in the second jaw 102 described above. For example, the cartridge 500 may be accommodated on a side closer to the distal end 100d than to the rotation shaft.

For example, the first jaw 101 may be formed entirely in the form of a hollow box with one surface (upper surface) thereof is removed, such that a cartridge accommodation part 101a capable of accommodating the cartridge 500 is formed inside the first jaw 101. That is, the first jaw 101 may be formed in an approximately “U” shape in cross section.

The guide groove 101h may be formed in a bottom surface of the first jaw 101, the bottom surface opposite to an upper open region formed by removing one surface. In some embodiments, the guide groove 101h may be configured to guide a linear motion of the operation member 140.

Referring to FIG. 4, the guide groove 101h may be configured to guide the operation member 140, and may have a through-hole shape in a region facing the operation member 140. Through this, one region of the operation member 140, such as at least one region of the operation member 140, or the second clamp 147 connected thereto may pass through the guide groove 101h and be ejected to the outside of the first jaw 101. When the operation member 140 moves forward, the second clamp 147 may pass through the guide groove 101h of the first jaw 101 and be exposed to the outside of the first jaw 101, and may come into contact with the lower surface of the first jaw 101 or apply pressure thereto. As the operation member 140 moves, the second clamp 147 applies pressure on the lower surface of the first jaw 101 and the first clamp 146 applies pressure on the upper surface of the second jaw 102 such that a gap between the second jaw 102 and the first jaw 101 decreases, allowing the second jaw 102 to naturally remain in a closed state with respect to the first jaw 101.

In an optional embodiment, the first jaw 101 may include a window 101b. After operating the operation member 140 or using the end tool 100, the second clamp 147 of the operation member 140 may be positioned corresponding to the window 101b, and the coupled state of the first jaw 101 and the operation member 140 may be released.

The plurality of fixed pulleys 120 may be disposed in front of the cartridge accommodation part 101a of the first jaw 101, and, for example, the first fixed pulley 121 and the second fixed pulley 122 may be disposed.

The first fixed pulley 121 and the second fixed pulley 122 may be disposed to have an inclined shape rather than being disposed parallel to each other. For example, the first fixed pulley 121 and the second fixed pulley 122 may be disposed such that a distance therebetween decreases in a direction away from the second jaw 102.

In some embodiments, the first fixed pulley 121 and the second fixed pulley 122 may be disposed in the first jaw 101 to face each other, and, for example, the first fixed pulley 121 and the second fixed pulley 122 may be disposed symmetrically to each other. For example, the first fixed pulley 121 and the second fixed pulley 122 may have the same size.

The first advancing wire 111 and the second advancing wire 112 may be correspondingly wound around the first fixed pulley 121 and the second fixed pulley 122, and the regions of the first advancing wire 111 and the second advancing wire 112 emerging from being wound around the lower sides of the first fixed pulley 121 and the second fixed pulley 122 may be directed to the operation member 140 described above.

With such a shape, the balanced arrangement of the first and second fixed pulleys 121 and 122 and the first and second advancing wires 111 and 112 relative to the moving direction of the operation member 140 may be enhanced, allowing for easier implementation of a symmetrical shape. For example, shaking or rotational moment generated when pulling the first advancing wire 111 and the second advancing wire 112 may be reduced, thereby minimizing or preventing shaking of the end tool 100.

For example, the end tool 100 may be shaped to reduce a width of, for example, one side of the first jaw 101, which forms one side of the jaw 103, specifically a width of a main region on the lower side, thereby allowing the end tool 100 to be implemented with a compact overall structure.

For example, the first fixed pulley 121 and the second fixed pulley 122 may be disposed to have an inclined shape rather than being positioned parallel to each other, so that certain regions of the two advancing wires 111 and 112 are wound downward around the first fixed pulley 121 and the second fixed pulley 122 and then connected to the operation member 140, while other regions of the two advancing wires 111 and 112 are wound upward around the first fixed pulley 121 and the second fixed pulley 122 and positioned on opposite sides of the cartridge 500, that is, the advancing wires 111 and 112 are disposed between the cartridge 500 and opposite side surfaces of the first jaw 101, respectively.

Further, by arranging the two advancing wires 111 and 112 correspondingly to the first fixed pulley 121 and the second fixed pulley 122 formed in an inclined shape, such as a symmetrical shape, maximum tensions applied to the two advancing wires 111 and 112 can be equalized or made almost similar to each other, this may enhance the fatigue life of each wire.

Further, one regions of the two advancing wires 111 and 112 can be controlled to symmetrically pass through the lower side of the cartridge 500, and another regions thereof can be controlled to symmetrically pass through opposite sides of the cartridge 500, and accordingly, unwanted moment or rotational forces generated when pulling the two advancing wires 111 and 112 may be reduced or prevented from causing unintended movement or shaking of the end tool 100 or the surgical instrument 10 including the same.

The first fixed pulley 121 and the second fixed pulley 122 may be disposed to have an inclined shape, i.e., a slanted shape, rather than being disposed parallel to each other.

For example, each of the first fixed pulley 121 and the second fixed pulley 122 may be disposed to be inclined at an angle of 35 degrees (deg) to 51 degrees (deg) relative to a vertical line (a line parallel to a Z-axis) based on FIG. 6.

Further, in other words, a central axis of the first fixed pulley 121 and a central axis of the second fixed pulley 122 may be inclined at an angle without being parallel to each other, and for example, the central axis of the first fixed pulley 121 and the central axis of the second fixed pulley 122 may be disposed to be inclined at an angle of 78 degrees to 110 degrees.

Further, in other words, as shown in FIG. 6, the first fixed pulley 121 and the second fixed pulley 122 may be shaped to be inclined, forming an adjacent angle.

The adjacent angle may range from 70 degrees (deg) to 112 degrees (deg).

When the adjacent angle is less than 70 degrees or more than 112 degrees, a diameter of each of the first fixed pulley 121 and the second fixed pulley 122 accommodated in a predetermined space of the first jaw 101 becomes smaller, which causes the stress applied on the advancing wires 111 and 112 to be increased, and the maximum allowable tension and durability of the advancing wires 111 and 112 to be reduced, and thus, the adjacent angle may range from 35 degrees (deg) to 51 degrees (deg), in a specific example, the adjacent angle may be 86 degrees (deg). For example, the adjacent angle is applicable to embodiments to be described below without change.

The arrangement relationship of the operation member 140, the plurality of advancing wires 110, and the plurality of fixed pulleys 120 will now be further described.

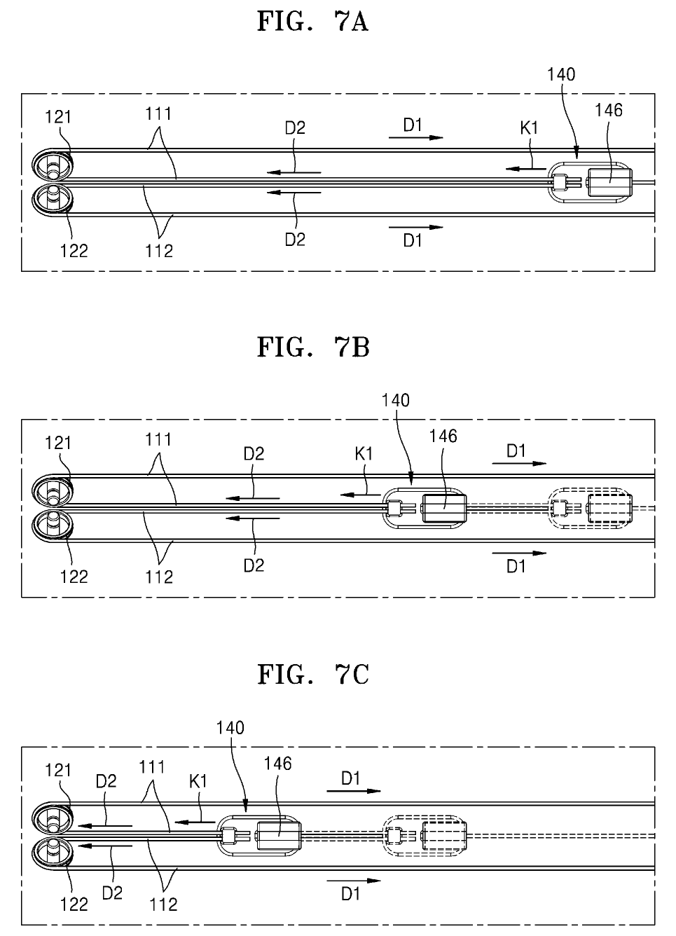

FIGS. 7A to 7C are schematic views for describing operations of the operation member of the end tool of FIG. 6.

Referring to FIGS. 7A to 7C, for convenience of description, the first jaw 101 is excluded, and the first advancing wire 111, the second advancing wire 112, the first fixed pulley 121, the second fixed pulley 122, and the operation member 140 are illustrated.

Based on FIGS. 7A to 7C, the operation member 140 may move in a leftward direction, i.e., move forward toward the distal end 100d, and this forward movement is illustrated sequentially in FIGS. 7A, 7B, and 7C.

As shown in FIG. 7A, when the first advancing wire 111 and the second advancing wire 112 are pulled in a first direction D1, one region of each of the first advancing wire 111 and the second advancing wire 112 is pulled in the first direction D1, and thus the regions of the first advancing wire 111 and the second advancing wire 112 emerging from the lower sides of the first fixed pulley 121 and the second fixed pulley 122 after being wound around the upper sides thereof move in a second direction D2, which is the opposite direction of the first direction D1. Accordingly, the operation member 140 also moves in a direction K1, which is the same direction as the second direction D2, i.e., move forward, thereby positioning the operation member 140 in an advanced position shown in FIG. 7B.

Thereafter, as shown in FIG. 7A, when the first advancing wire 111 and the second advancing wire 112 are pulled further in the first direction D1, one region of each of the first advancing wire 111 and the second advancing wire 112 is pulled further in the first direction D1, and thus the regions of the first advancing wire 111 and the second advancing wire 112 emerging from the lower sides of the first fixed pulley 121 and the second fixed pulley 122 after being wound around the upper sides thereof further move in the second direction D2, which is the opposite direction of the first direction D1. Accordingly, the operation member 140 further moves in the direction K1, which is the same direction as the second direction D2, that is, advances to a position further forward than that shown in FIG. 7B, and as illustrated in FIG. 7C, the operation member 140 advances to a position further forward than that shown in FIG. 7B.

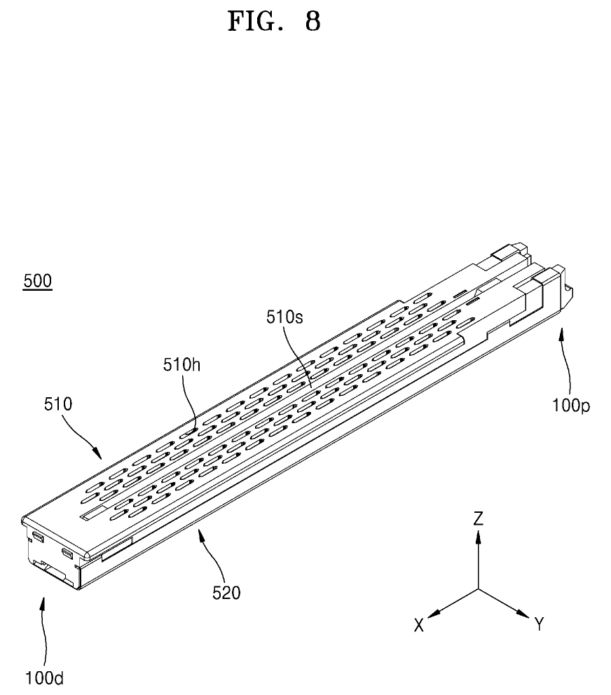

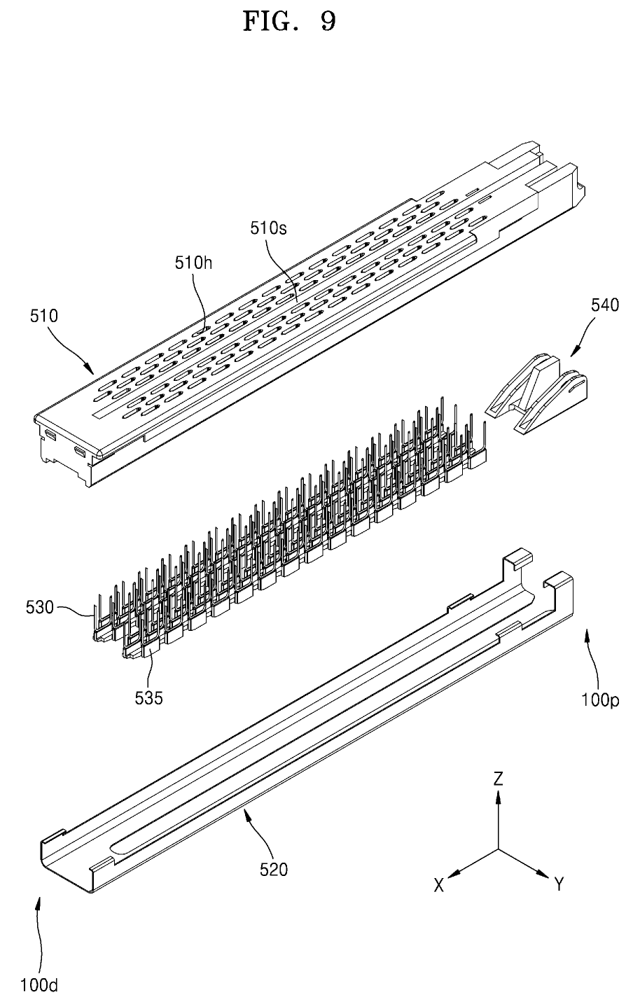

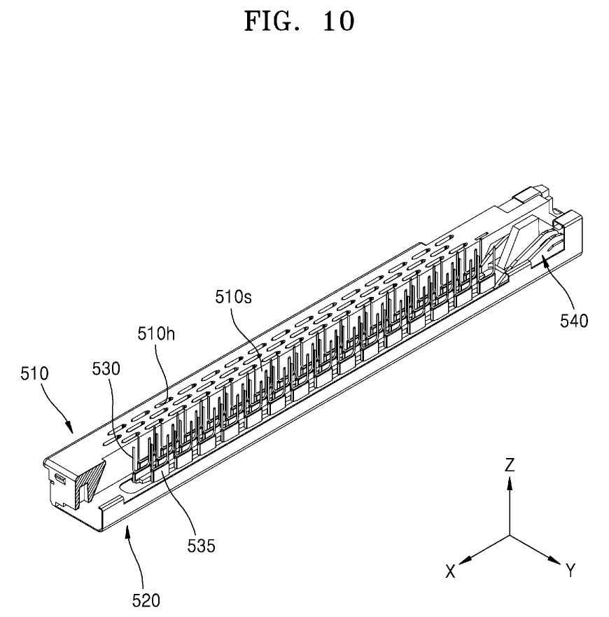

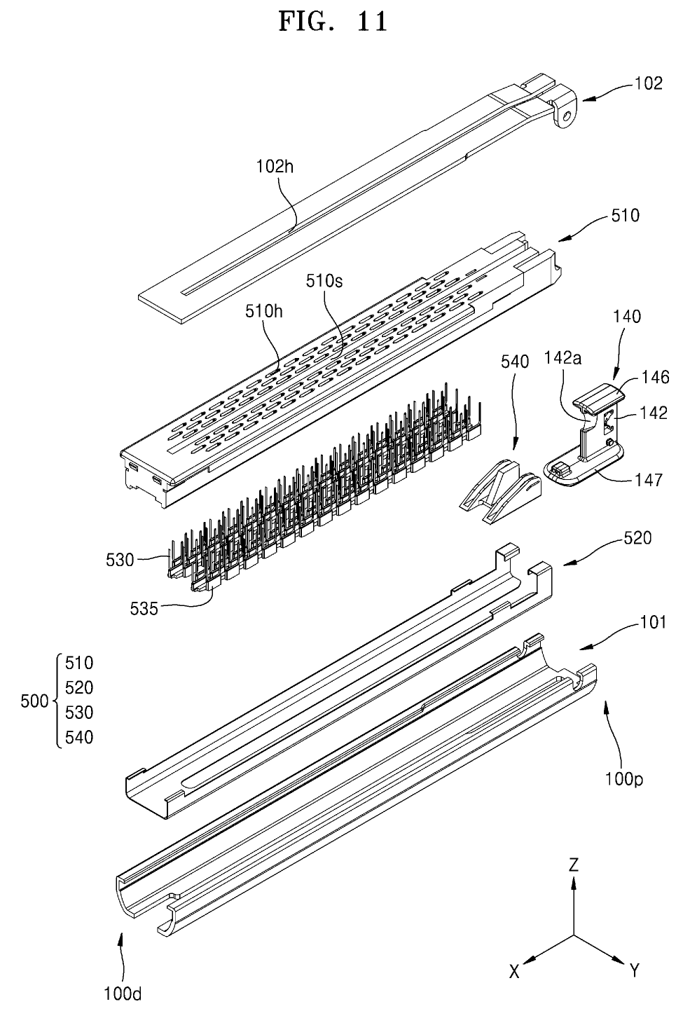

FIG. 8 is a perspective view of the cartridge of FIG. 3, FIG. 9 is an exploded perspective view of the cartridge of FIG. 8, FIG. 10 is a perspective view of the cartridge of FIG. 8 with a portion of a case of the cartridge removed, and FIG. 11 is an exploded perspective view illustrating the end tool and the cartridge of FIG. 3.

The cartridge 500 may be positioned in the first jaw 101, and for example, the cartridge 500 may be positioned by being coupled to the cartridge accommodation part 101a of the first jaw 101. For example, the cartridge 500 may be integrally formed with the first jaw 101 while the operation member 140 is positioned in the first jaw 101. Further, in an optional embodiment, the cartridge 500 may be configured to be mountable to and dismountable from the first jaw 101.

The cartridge 500 includes the plurality of staples 530 therein to perform suturing of tissue, and performs cutting through the operation member 140. Here, the cartridge 500 may include a cover 510, the housing 520, the staples 530, withdrawal members 535, and the moving member 540.

The cover 510 may be configured to cover an upper portion of the cartridge accommodation part 101a of the first jaw 101. Staple holes 510s through which the plurality of staples 530 may be ejected to the outside may be formed in the cover 510. As the staples 530, which are accommodated inside the cartridge accommodation part 101a before a stapling operation, are pushed and raised upward by the operation member 140 during a stapling motion, and pass through the staple holes 510s of the cover 510 to be withdrawn out of the cartridge 500, stapling may be performed.

For example, the slit 510s may be formed in the cover 510 along a longitudinal direction of the cover 510. As shown in FIG. 5, the blade 142a of the operation member 140 may protrude to the outside of the cartridge 500 through the slit 510s. As the blade 142 of the operation member 140 passes along the slit 510s, staple-completed tissue may be cut.

The cartridge 500 may include the housing 520, and the staples 530 and the moving member 540 may be positioned in the housing 520 after the housing 520 is positioned in the accommodation part 101a of the first jaw 101, and the cover 510 may be configured to cover an upper portion of the housing 520.

The housing 520 forms an outer shape of the cartridge 500, and may be formed entirely in the form of a hollow box with one surface (upper surface) thereof removed so that the staples 530 and the moving member 540 are accommodated in the hollow box. That is, the housing 520 may be formed in a substantially “U” shape in cross section.

The plurality of staples 530 may be positioned inside the cartridge accommodation part 101a of the first jaw 101. As the operation member 140 linearly moves in one direction, the plurality of staples 530 are sequentially pushed and raised from the inside of the cartridge accommodation part 101a of the first jaw 101 to the outside, thereby performing suturing, that is, stapling. Here, the staples 530 may include a material that is durable and does not have an abnormal effect on the human body, such as titanium, stainless steel, or the like.

For example, the withdrawal members 535 may be further disposed between the cartridge accommodation part 101a of the first jaw 101 and the staples 530. In other words, it may be said that the staple 530 is positioned above the withdrawal member 535. In this case, the operation member 140 is linearly moved in one direction to push the withdrawal member 535 upward, and the withdrawal member 535 may push the staple 530 upward.

As such, the operation member 140 may be described as pushing the staples 530 upward in both the case in which the operation member 140 directly pushes the staples 530 upward and the case in which the operation member 140 pushes the withdrawal members 535 upward and the withdrawal members 535 pushes the staples 530 upward (i.e., the operation member 140 indirectly pushes the staples 530 upward).

As described above, the operation member 140 may be positioned inside the cartridge accommodation part 101a of the first jaw 101. Further, the operation member 140 may include the moving member 540 or may be used together with the moving member 540, and when the operation member 140 moves, the moving member 540 may move together with the operation member 140, and as a result, the moving member 540 may directly push the staples 530 upward or push the withdrawal members 535 upward, which in turn pushes the staples 530 upward.

As described above, as the advancing wire 110 moves, i.e., the advancing wire 110 is pulled, the operation member 140 connected thereto may move forward toward the distal end 100d of the first jaw 101.

Such a forward movement of the operation member 140 may cause the moving member 540 to push the withdrawal member 535 upward, which may also cause the staple 530 to rise, and at the same time, cutting using the blade 142a of the operation member 140 may be performed. Further, in an optional embodiment, in the case of the end tool 100 in which a retracting wire is connected to the operation member 140, the retracting wire may be pulled to cause the operation member 140 to move backward toward the proximal end 100p of the first jaw 101.

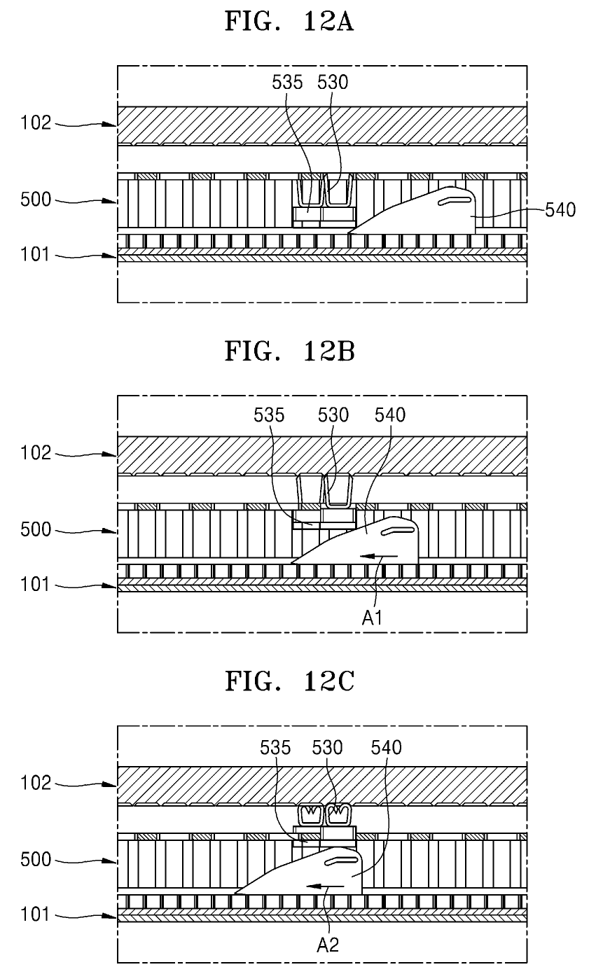

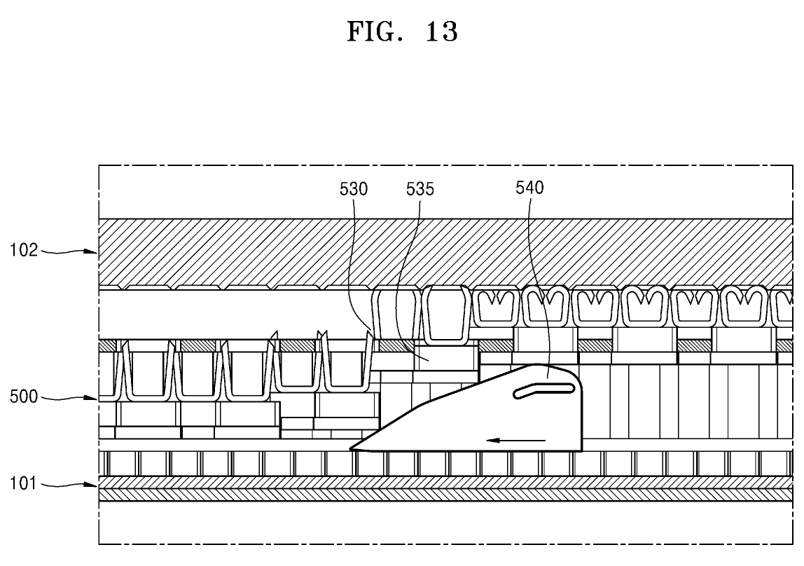

FIGS. 12A to 13 are cross-sectional views illustrating an overall staple operation of the end tool of the surgical instrument of FIG. 1.

Referring to FIGS. 12A to 13, in the state shown in FIG. 12A, as the operation member 140 moves in the direction of an arrow A1 in FIG. 12B, the moving member 540, specifically the inclined surface of the moving member 540, pushes the withdrawal member 535 upward, and the withdrawal member 535 pushes one side of a lower portion of the staple 530 upward. For example, due thereto, the staple 530 is ejected to the outside of the first jaw 101 and the cartridge 500.

In this state, when the operation member 140 further moves in the direction of an arrow A2 of FIG. 12C, the ejected staple 530 continues to be pushed upward by the operation member 140 while in contact with the lower surface of the second jaw 102, such as an anvil, so that stapling is performed while both end portions of the staple 530 are bent.

As such motions are continuously performed, stapling may be sequentially performed from the staple 530 on the proximal end 100p side (see FIG. 3) to the staple 530 on the distal end 100d side (see FIG. 3) among the plurality of staples 530, as illustrated in FIG. 13.

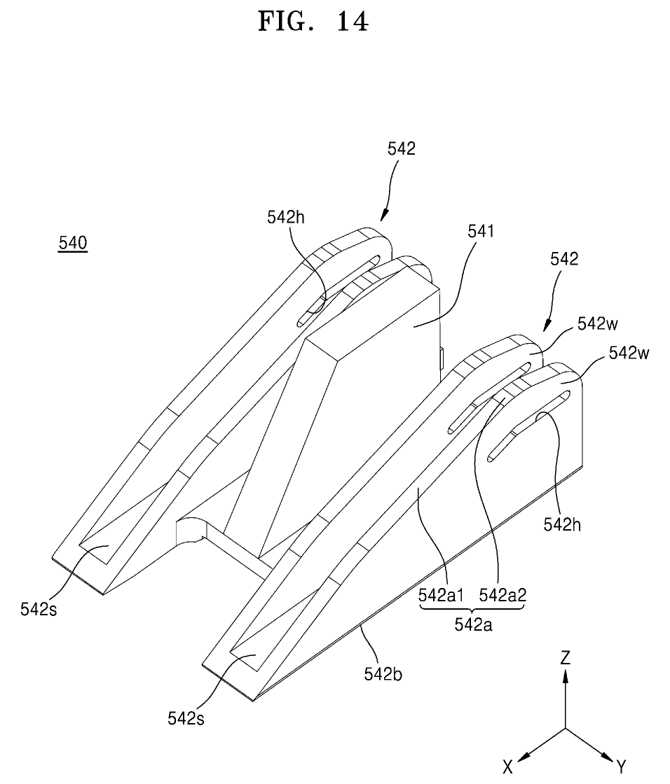

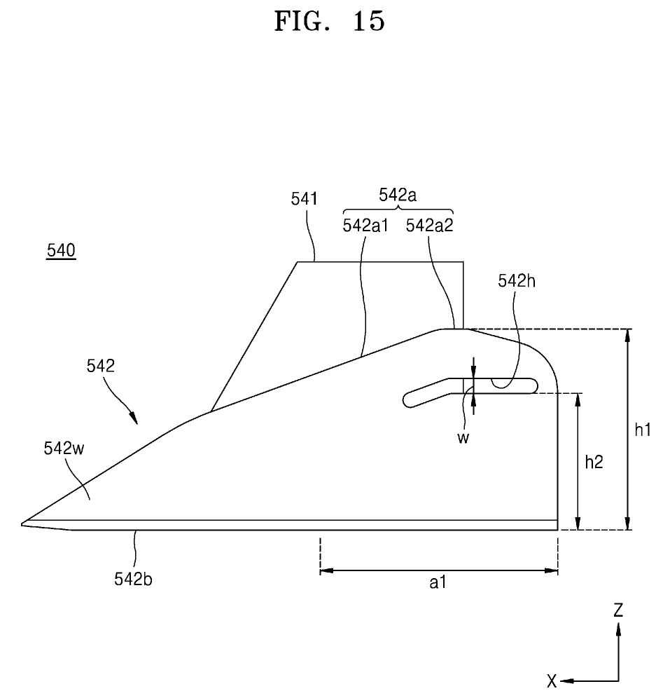

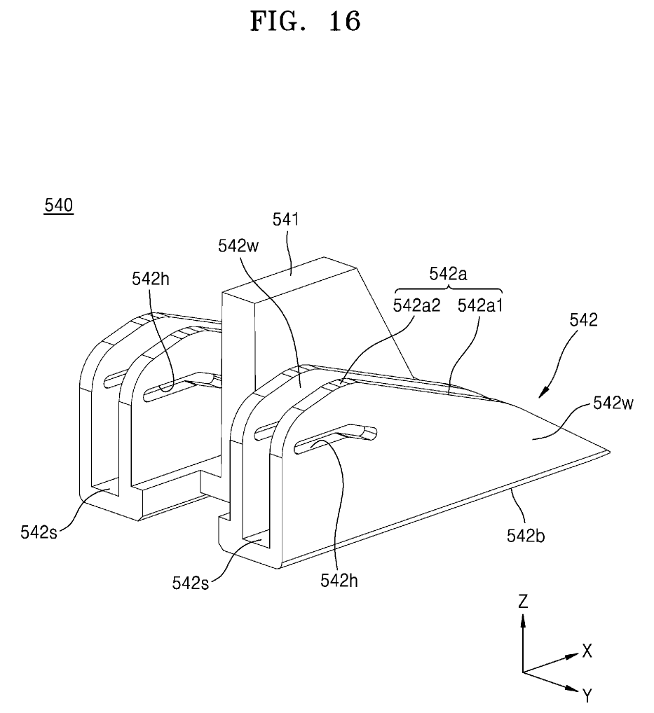

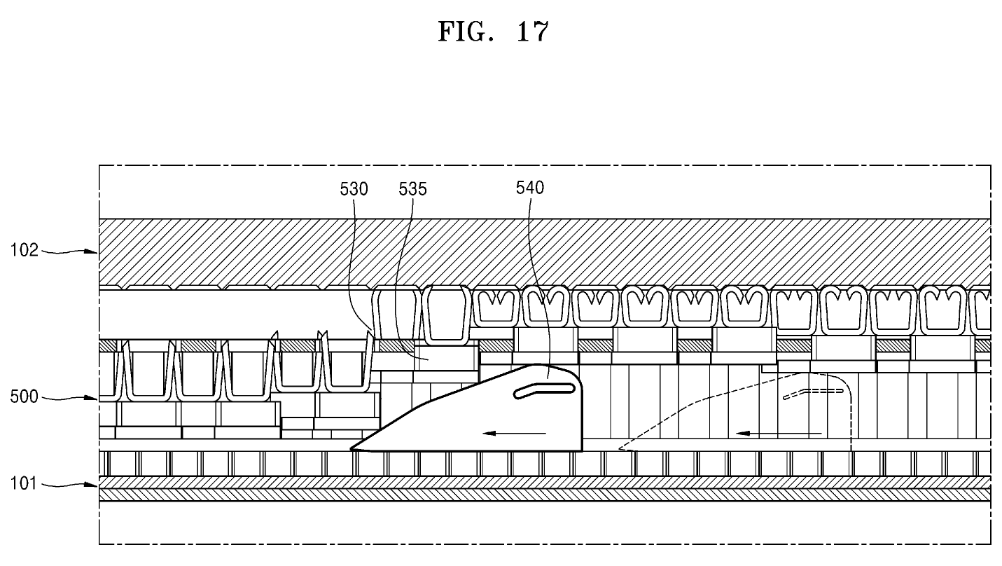

FIG. 14 is a perspective view of the moving member of the cartridge of FIG. 9, FIG. 15 is a side view of the moving member of FIG. 14, FIG. 16 is a perspective view of the moving member of FIG. 14, viewed from another direction, and FIG. 17 is a cross-sectional view illustrating an overall staple operation of the moving member of FIG. 14.

Referring to FIGS. 14 to 16, the moving member 540 may include a region, which is deformable according to an external force and pushes the staple 530 upward in response to the movement of the operation member 140. In other words, the region deformable according to an external force may also be described as a region that is less rigid than other regions.

In describing the present disclosure, for convenience of description, the region of the moving member 540 that is deformable according to an external force is defined as a first region a1.

Referring to FIGS. 10 and 14 to 16, the moving member 540 may be positioned inside the housing 520 and may move along the slit 510s relative to the housing 520.

The moving member 540 may include a body part 541 and wedge parts 542. The body part 541 is positioned in the slit 510s and may move along the slit 510s relative to the housing 520. In other words, the body part 541 may be guided along the path defined by the slit 510s, which extends in the first direction, and may move from the proximal end 100p of the first jaw 101 to the distal end 100d, or from the distal end 100d to the proximal end 100p.

The wedge parts 542 may be positioned on opposite sides of the body part 541, and may push the plurality of staples 530 upward. The plurality of staples 530 may each be positioned on opposite sides of the slit 510s, and when the moving member 540 moves along the slit 510s, the wedge parts 542, which are positioned on opposite sides of the body part 541, may push each of the plurality of staples 530, which are positioned on opposite sides of the slit 510s, upward.

The wedge part 542 positioned on one side of the body part 541 may include a pair of wedges 542w. The pair of wedges 542w may be connected by a support 542s. In other words, the pair of wedges 542w may be spaced apart by a preset distance by the support 542s.

Accordingly, as shown in FIG. 14, one moving member 540 may have a total of four wedges 542w formed thereon.

Among the four wedges 542w, two wedges 542w may be paired and positioned on one side of the body part 541, while the remaining two wedges 542w may also be paired and positioned on another side of the body part 541.

A spacing between the pair of wedges 542w positioned on one side of the body part 541 may be the same as a width of the support 542s. As the pair of wedges 542w are positioned at a preset spacing by the support 542s, the wedge parts 542 may be in contact with the withdrawal members 535 or the staples 530 with a larger surface area. As a result, the wedge parts 542 may simultaneously push multiple staples 530 upward and withdraw the staples 530 to the outside of the first jaw 101.

However, the present disclosure is not limited thereto, and various modifications may be made within the technical concept of pushing multiple staples 530 upward and withdrawing the plurality of staples 530 externally. For example, the wedge part 542 positioned on one side of the body part 541 may include three or more wedges 542w positioned at a preset spacing.

The wedge part 542 may include a contact surface 542a and a bottom surface 542b.

The contact surface 542a may be a surface that comes into contact with the staples 530 when pushing the plurality of staples 530 upward. The contact surface 542a may have a predetermined incline. That is, the wedge part 542 may be inclined to a certain degree with respect to the first direction, which is a direction in which the housing 520 extends. The contact surface 542a may be disposed on each of the wedges 542w. Thus, the contact surface 542a may be provided in a quantity corresponding to the number of wedges 542w.

Referring to FIGS. 14 to 16, the bottom surface 542b may be one surface of the wedge part 542 opposite to the contact surface 542a. The bottom surface 542b may be in contact with the housing 520 when the wedge part 542 moves relative to the housing 520. In other words, the bottom surface 542b may be located at the first jaw 101 when the wedge part 542 moves.

Further, the bottom surface 542b may be one surface of the support 542s facing the housing 520.

The contact surface 542a may include an inclined surface 542a1 and an operating surface 542a2. The inclined surface 542a1 may be a surface that has a predetermined incline with respect to the housing 520.

That is, the inclined surface 542a1 may be inclined to a certain degree in a direction in which the end tool 100 extends or the first direction. In other words, the inclined surface 542a1 may be formed such that a height of the portion thereof adjacent to the proximal end 100p of the first jaw 101 is greater than a height of the portion thereof adjacent to the distal end 100d of the first jaw 101.

The inclined surface 542a1 may be positioned to have a preset inclination angle with respect to the housing 520. For example, the inclined surface 542a1 may form a dual-incline shape, in which the inclination angle transitions at a specific point. In an optional embodiment, the inclined surface 542a1 may bend at the middle thereof, transitioning from a gentle slope to a steep slope, or vice versa, transitioning from a steep slope to a gentle slope.

The operating surface 542a2 is connected to the inclined surface 542a1, and may be a surface where the wedge part 542 reaches its maximum height relative to the bottom surface 542b. The wedge part 542 may gradually increase in height from the inclined surface 542a1 toward the operating surface 542a2. In this case, the height of the wedge part 542 may reach its maximum at the operating surface 542a2, and beyond the operating surface 542a2, the height of the wedge part 542 may decrease again.

In the present specification, for convenience of description, the height of the operating surface 542a2 is defined as a wedge-part maximum height h1. The wedge-part maximum height h1 may refer to the height of the operating surface 542a2 based on the bottom surface 542b or the height the operating surface 542a2 in a Z-axis direction based on FIG. 15.

The wedge-part maximum height h1 may affect final formed heights of the staples 530 when the moving member 540 moves inside the housing 520 while pushing the plurality of staples 530 upward and withdrawing the staples 530.

The final formed height of the staples 530 refers to a height of the staples 530 after being pushed upward by the moving member 540, with both ends bent and the stapling process completed.

As shown in FIGS. 12A to 13, when the moving member 540 moves inside the housing 520 and pushes the plurality of staples 530 upward, the ejected staples 530 may be further pushed upward by the operation member 140 while in contact with the lower surface of the second jaw 102, such as the anvil, causing both end portions of the ejected staples 530 to bend.

When the wedge-part maximum height h1 is high, the withdrawn staples 530 may be lifted relatively higher, thereby reducing a distance between the lower surface of the second jaw 102 and the withdrawal member 535, and resulting in the final height of the staples 530 being formed relatively lower.

Conversely, when the wedge-part maximum height h1 is low, the withdrawn staples 530 may be lifted relatively less, thereby increasing the distance between the lower surface of the second jaw 102 and the withdrawal member 535, and resulting in the final height of the staples 530 being formed relatively higher.

In other words, when the wedge-part maximum height h1 is high, the final formed height of the staple 530 may be relatively low, and when the wedge-part maximum height h1 is low, the final formed height of the staple 530 may be relatively high.

The cartridge 500 according to an embodiment of the present disclosure employs the moving member 540 having a region deformable according to an external force, thereby enabling the final formed heights of the staples 530 to be appropriately adjusted based on the thickness of the body tissue on which the stapling is performed.

Hereinafter, the first region a1 of the moving member 540 will be described in more detail.

Referring to FIGS. 14 to 16, the first region a1 may be positioned in the wedge part 542. At this time, a height of the moving member 540 may be adjusted as the contact surface 542a of the wedge part 542 is deformed by an external force.

A wedge hole 542h, which is positioned at a preset height of the first region a1 and extends in the first direction, may be formed in the wedge part 542 of the moving member 540.

In the present specification, the height from the bottom surface 542b to the wedge hole 542h is defined as a wedge-hole height h2.

The wedge hole 542h may have a constant width w. When the wedge part 542 receives an external force directed from the contact surface 542a toward the bottom surface 542b, the first region a1 may be pressed by the external force directed toward the bottom surface 542b and partially deformed. In some embodiments, the inclined surface 542a1 and the operating surface 542a2 of the first region a1 may be pressed toward the wedge hole 542h and deformed.

The maximum deformation amount of the first region a1 may be determined by the width w of the wedge hole 542h. For example, the maximum deformation amount of the first region a1 may be substantially equal to the width w of the wedge hole 542h. Thus, the wedge-part maximum height h1 may be adjusted within the range defined by the width w of the wedge hole 542h.

The first region a1 may have an elastic restoring force. Accordingly, even when the first region a1 is partially deformed by an external force, the first region a1 may return to its original shape once the external force is removed.

Referring to FIG. 15, at least a portion of the wedge hole 542h may be formed in a direction parallel to the inclined surface 542a1. As a result, the wedge hole 542h may be formed in a shape that extends in the first direction and bends partially at a certain point. In an optional embodiment, the point at which the wedge hole 542h is bent may be formed at a position corresponding to a point at which the inclined surface 542a1 and the operating surface 542a2 are connected.

The wedge part 542 may include first end positioned toward the distal end 100d of the cartridge 500 and second end positioned toward the proximal end 100p of the cartridge 500, and the wedge hole 542h may be positioned closer to the second end than to the first end. In other words, the wedge hole 542h may be positioned closer to the rear end (right side based on FIG. 15) of the wedge part 542.

Referring to FIG. 15, the wedge part 542 increases in height from the distal end 100d of the cartridge 500 toward the proximal end 100p of the cartridge 500, and the operating surface 542a2, which determines the wedge-part maximum height h1, may also be positioned closer to the proximal end 100p of the cartridge 500, i.e., the rear end of the wedge part 542.

As the wedge hole 542h is positioned adjacent to the operating surface 542a2, the first region a1 may be easily deformed by an external force, and the wedge-part maximum height h1 may also be easily adjusted by the external force.

Referring to FIGS. 15 and 16, the wedge hole 542h may be positioned closer to the contact surface 542a than to the bottom surface 542b. In other words, it may also be said that the wedge hole 542h is positioned closer to the cover 510 than to the housing 520 of the cartridge 500.

In an optional embodiment, the wedge-hole height h2 may have a value greater than half of the wedge-part maximum height h1.

When the wedge hole 542h is positioned at an upper portion of the wedge part 542 (upper side based on FIG. 15), a region between the contact surface 542a and the wedge hole 542h becomes narrower. As a result, when an external force is applied to the wedge part 542, stress may be concentrated around the wedge hole 542h, thereby allowing the wedge-part maximum height h1 to be easily adjusted.

For example, when the wedge hole 542h is positioned at a lower portion of the wedge part 542, the external force may be dispersed in the process of being transmitted to the lower portion of the wedge part 542, which may result in a reduction in the deformation amount of the wedge part 542. Further, the region between the contact surface 542a and the wedge hole 542h may become wider, and thus leading to the issue of the deformed wedge part 542 failing to return to its original shape and undergoing permanent deformation.

Referring to FIGS. 14 and 16, the pair of wedges 542w of the wedge part 542 positioned on one side of the body part 541 may be symmetrical. In other words, the wedges 542w facing each other may be symmetrical. Accordingly, the wedge holes 542h facing each other may also be symmetrical.

Accordingly, when an external force is applied to the wedge part 542, the external force may be evenly distributed across the pair of wedges 542w, and the amount of deformation of the wedge part 542 due to the external force may also be the same. In other words, the pair of wedges 542w may push the staples 530 upward to the same height.

In an optional embodiment, each of the wedge holes 542h formed in the pair of wedges 542w facing each other may have a different shape rather than being symmetrical. In some embodiments, the width w of one of the wedge holes 542h facing each other may be greater than that of another one of the wedge holes 542h. For example, the wedge-hole heights h2 of a plurality of wedge holes 542h facing each other may be different from each other.

As a result, when a plurality of wedges 542w push different withdrawal members 535 upward, respectively, the amount of deformation of each wedge 542w may vary, and the formed heights of the withdrawn staples 530 may also be different.

A description is provided for the case in which the moving member 540 pushes the withdrawal member 535 upward, thereby pushing the staple 530 upward.

When the plurality of staples 530 are positioned on a single withdrawal member 535, the plurality of staples 530 may be pushed upward by the single withdrawal member 535, and thus, may have the same final formed height.

For example, when the withdrawal member 535 is divided such that two staples 530 are positioned on one withdrawal member 535 and one staple 530 is positioned on another withdrawal member 535, the final formed heights of the staples 530 pushed upward by each withdrawal member 535 may also be different.

The amount of deformation of the wedge part 542 may vary depending on the magnitude of an external force applied to the wedge part 542. When a relatively large external force is applied to the wedge part 542, the wedge part 542 undergoes greater deformation, whereas when a relatively small external force is applied to the wedge part 542, the wedge part 542 either does not deform or deforms to a lesser extent.

When the external force applied to the wedge part 542 is removed, the wedge hole 542h may return to its original state due to the elastic restoring force of the wedge part 542.

In other words, the wedge-part maximum height h1 may be adjusted according to an external force applied to the wedge part 542.

Referring to FIG. 12, as the operation member 140 moves in the direction of the arrow A1 in FIG. 12B, the moving member 540, specifically the inclined surface of the moving member 540, pushes the withdrawal member 535 upward, and the withdrawal member 535 pushes one side of the lower portion of the staple 530 upward. For example, due thereto, the staple 530 is ejected to the outside of the first jaw 101 and the cartridge 500.

As such motions are continuously performed, as shown in FIG. 13, stapling may be sequentially performed from the staples 530 located on the proximal end 100p side (see FIG. 3) to the staples 530 located on the distal end 100d side (see FIG. 3) among the plurality of staples 530.

The shape of the moving member 540 shown as a solid line on the left side of FIG. 17 illustrates a case in which the body tissue on which stapling is performed is relatively thin, while the shape of the moving member 540 shown as a dotted line on the right side of FIG. 17 illustrates a case in which the body tissue is relatively thick.

Hereinafter, the stapling process shown in FIG. 17 will be described in detail.

The external force applied to the wedge part 542 may be determined according to the thickness of the body tissue positioned between the first jaw 101 and the second jaw 102. In some embodiments, during the stapling process using the surgical instrument 10, a reactive force may be generated in the body tissue positioned between the cartridge 500 and the second jaw 102 due to a pressure applied vertically. In this case, as the thickness of the body tissue increases, a greater reactive force is generated.

The reactive force dependent on the thickness of the body tissue may act as an external force on the wedge part 542. As the thickness of the body tissue increases, the reactive force also increases, and thus, the wedge part 542 undergoes greater deformation, and consequently, the wedge-part maximum height h1 decreases.

As a result, the withdrawal member 535 is pushed upward to a relatively lower height, and the final formed height of the staple 530 ejected to the outside of the first jaw 101 and the cartridge 500 may be relatively higher.

In other words, as the body tissue on which stapling is performed becomes thicker, the final formed height of the staple 530 becomes relatively higher, and conversely, as the body tissue becomes thinner, the final formed height of the staple 530 becomes relatively lower.

In the cartridge 500 according to an embodiment of the present disclosure, the moving member 540 employed in the cartridge 500 includes the first region a1 that is deformable by an external force, thereby allowing the final formed heights of the staples 530 to be appropriately adjusted according to the thickness of the body tissue on which stapling is performed.

Further, as the final formed height of each staple 530 is individually adjusted according to variations in the thickness of the body tissue, when the thickness of the body tissue within a single stapling section changes, the final formed heights of the staples 530 may naturally change in accordance with the thickness variation.

Further, even when the thicknesses of the body tissues positioned on opposite sides of the body part 541 are different, the heights of the wedge parts 542 positioned on opposite sides of the body part 541 may be individually adjusted according to the respective thicknesses, thereby allowing the final formed heights of the staples 530 to be individually set to match the thicknesses of the body tissues on opposite sides of the body part 541.

Further, even when the thickness of the body tissue positioned on one side of the body part 541 varies in a direction away from the body part 541, the heights of the plurality of wedges 542w positioned on one side of the body part 541 may be individually adjusted according to the varying thickness, thereby allowing the final formed heights of the staples 530 to be individually set based on the thickness variation of the body tissue positioned on one side of the body part 541.

Hereinafter, a configuration of a moving member according to another embodiment of the present disclosure will be described.

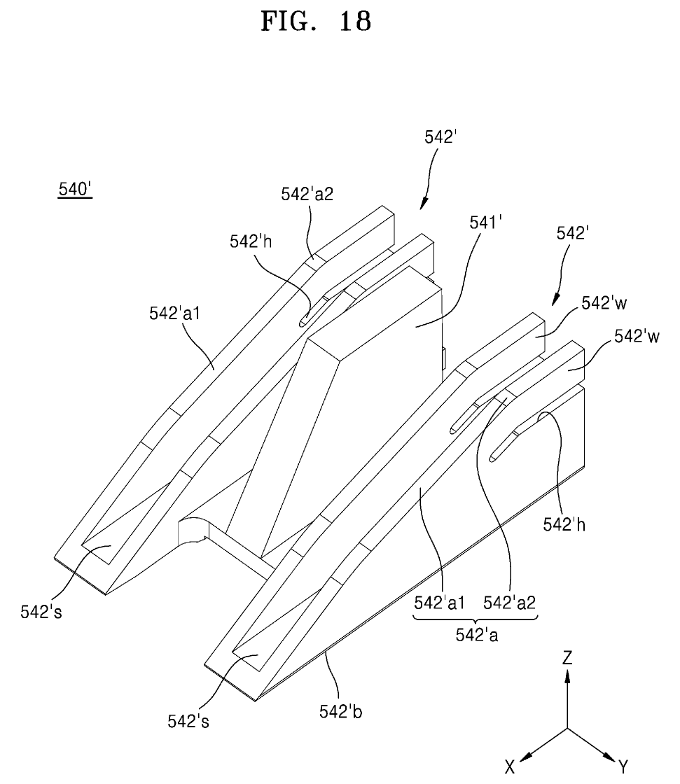

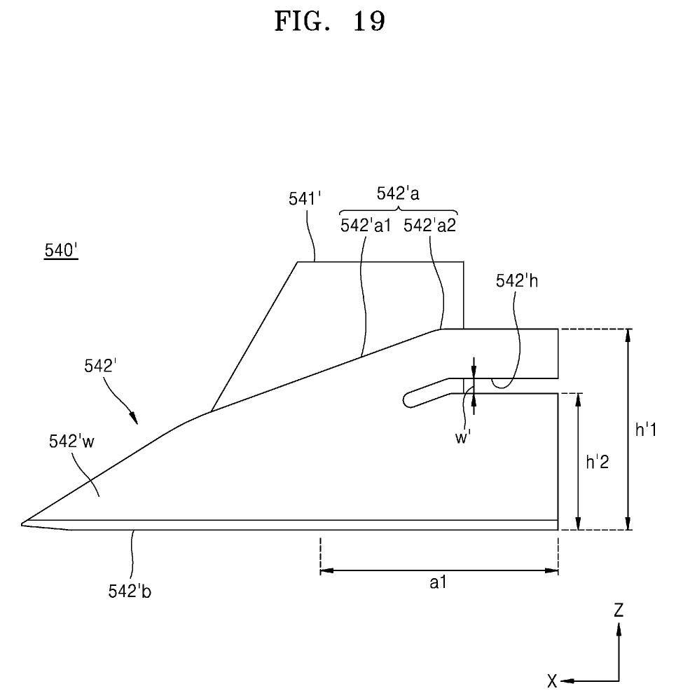

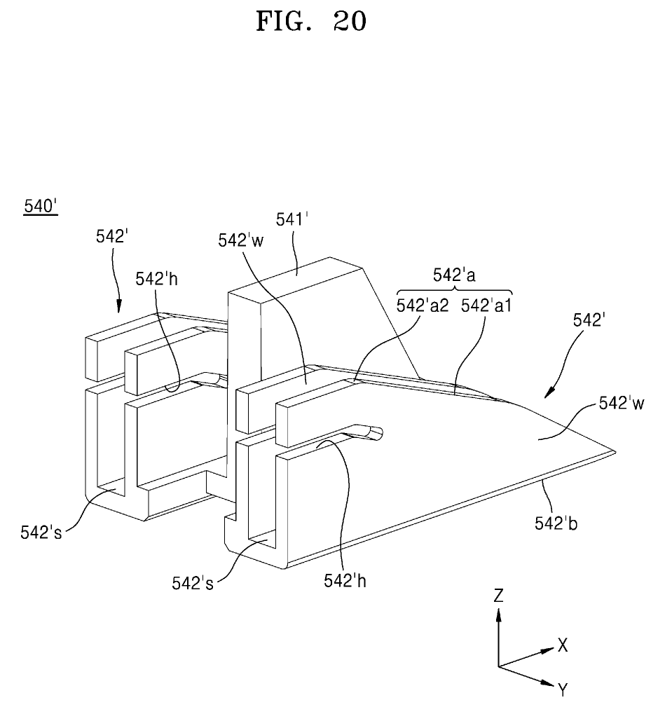

FIG. 18 is a perspective view of the moving member according to another embodiment of the present disclosure, FIG. 19 is a side view of the moving member of FIG. 18, and FIG. 20 is a perspective view of the moving member of FIG. 18, viewed from another direction.

Referring to FIGS. 18 to 20, a moving member 540` according to another embodiment of the present disclosure may include a body part 541’ and wedge parts 542`.

The moving member 540` according to another embodiment of the present disclosure is different from the moving member 540 according to an embodiment of the present disclosure in that there is a difference in a wedge groove 542’h, and thus, a detailed description will be provided below with a focus on this difference.

The moving member 540` according to the present embodiment may be applied to various cartridges 500. For example, the moving member 540` may be applied to a cartridge 500 that includes a cover 510, a housing 520, and a plurality of staples 530, such as the cartridge 500 in the above-described embodiment, and may be applied as is or modified within a permissible range of equivalence, depending on the needs of other cartridges 500 with various structures.

Referring to FIGS. 18 to 20, a first region a1 may be positioned in the wedge part 542’. The wedge groove 542’h, which is positioned at a preset height of the first region a1 and open in a direction toward the proximal end 100p of the end tool 100, may be formed in the wedge part 542’ of the moving member 540`.

The wedge groove 542`h has a constant width w` and may extend in the first direction. An end portion of the wedge groove 542`h, which extends in the first direction and is positioned close to the proximal end 100p of the cartridge 500, may be open. Accordingly, when an external force is applied to the first region a1, a contact surface 542`a may be easily deformed.

A maximum deformation amount of the wedge part 542’ may be substantially equal to the width w` of the wedge groove 542`h. A wedge-part maximum height h’1 may be adjusted within the range defined by the width w’ of the wedge groove 542’h.

The first region a1 may have an elastic restoring force. Accordingly, even when the first region a1 is partially deformed by an external force, the first region a1 may return to its original shape once the external force is removed.

Referring to FIG. 19, at least a portion of the wedge groove 542’h may be formed in a direction parallel to an inclined surface 542’a1. The region formed parallel to the inclined surface 542’a1 may be positioned on a side opposite to the open end portion of the wedge groove 542`h.

In other words, the end portion of the wedge groove 542`h extending in the first direction may be open, and another end portion of the wedge groove 542`h extending in the direction parallel to the inclined surface 542’a1 may be formed closed.

The wedge groove 542`h may be formed in a shape that roughly extends in the first direction and bends partially at a certain point. In an optional embodiment, the point at which the wedge groove 542’h is bent may be formed at a position corresponding to the point at which the inclined surface 542’a1 and an operating surface 542’a2 are connected.

According to another embodiment of the present disclosure, the first region a1 of the wedge part 542’ may be configured to be parallel to the first direction. In some embodiments, the first region a1 of the wedge part 542’, which is positioned above the wedge groove 542’h (upper side based on FIG. 19), may be configured to be parallel to the wedge groove 542`h. In other words, it may be said that an upper portion of the first region a1 of the wedge part 542’ is configured to be parallel to a bottom surface 542’b of the wedge parts 542`.

The wedge part 542’ may include first end positioned toward the distal end 100d of the cartridge 500 and second end positioned toward the proximal end 100p of the cartridge 500, and the wedge groove 542’h may be positioned on the second end. In this case, the wedge groove 542’h may be connected to the second end of the wedge part 542’, and open toward the proximal end 100p of the cartridge 500.

Referring to FIG. 19, the wedge part 542’ increases in height from the distal end 100d of the cartridge 500 toward the proximal end 100p of the cartridge 500, and the operating surface 542’a2, which determines the wedge-part maximum height h’1, may also be positioned closer to the proximal end 100p of the cartridge 500, i.e., the rear end of the wedge part 542’.

As the wedge groove 542’h is positioned adjacent to the operating surface 542’a2, the first region a1 may be easily deformed by an external force, and the wedge-part maximum height h’1 may also be easily adjusted by the external force.

Referring to FIGS. 18 and 19, the wedge groove 542’h may be positioned closer to the contact surface 542’a than to the bottom surface 542’b. In other words, it may also be said that the wedge groove 542’h is positioned closer to the cover 510 than to the housing 520 of the cartridge 500.

In the present specification, the height from the bottom surface 542’b to the wedge groove 542’h is defined as a wedge-groove height h’2.

In an optional embodiment, the wedge-groove height h’2 may have a value greater than half of the wedge-part maximum height h’1.

When the wedge groove 542’h is positioned at an upper portion of the wedge part 542’ (the upper side based on FIG. 19), a region between the contact surface 542’a and the wedge groove 542’h becomes narrower. As a result, when an external force is applied to the wedge part 542’, stress may be concentrated around the wedge groove 542’h, thereby allowing the wedge-part maximum height h’1 to be easily adjusted.

For example, when the wedge groove 542’h is positioned at a lower portion of the wedge part 542’, the external force may be dispersed in the process of being transmitted to the lower portion of the wedge part 542’, which may result in a reduction in the deformation amount of the wedge part 542’. Further, the region between the contact surface 542’a and the wedge groove 542’h may become wider, and thus leading to the issue of the deformed wedge part 542’ failing to return to its original shape and undergoing permanent deformation.

The amount of deformation of the wedge part 542’ may vary depending on the magnitude of an external force applied to the wedge part 542’. When a relatively large external force is applied to the wedge part 542’, the wedge part 542’ undergoes greater deformation, whereas when a relatively small external force is applied to the wedge part 542’, the wedge part 542’ either does not deform or deforms to a lesser extent.

When the external force applied to the wedge part 542’ is removed, the wedge groove 542’h may return to its original state due to the elastic restoring force of the wedge part 542’.

In the moving member 540’ according to another embodiment of the present disclosure, the wedge-part maximum height h’1 may also be adjusted based on the thickness of the body tissue on which stapling is performed, as shown in FIG. 17, and as a result, formed heights of the staples 530 withdrawn from the cartridge 500 may be adjusted.

In other words, as the thickness of the body tissue increases, the formed height of the staple 530 increases, and as the thickness of the body tissue decreases, the formed height of the staple 530 decreases.

The moving member 540` according to another embodiment of the present disclosure has the same configuration, operating principle, and effect as the moving member 540 according to an embodiment of the present disclosure, except for the wedge groove 542`h, and accordingly, a detailed description thereof will be omitted in the overlapping range.

Hereinafter, a configuration of a moving member according to the other embodiment of the present disclosure will be described.

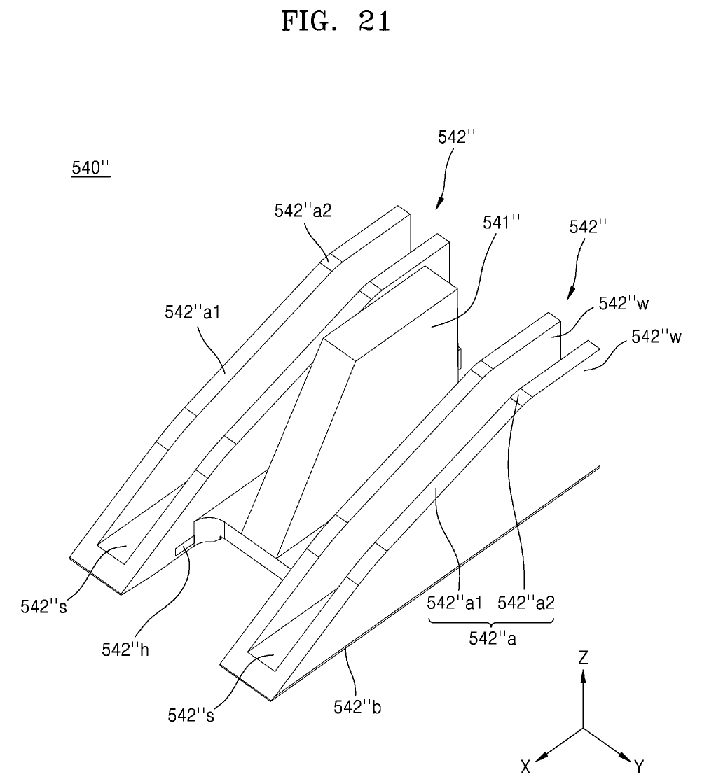

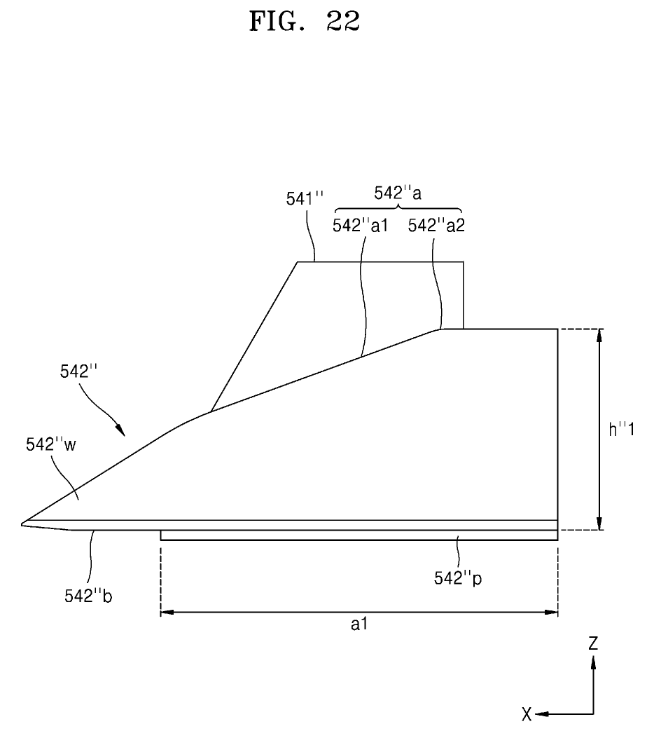

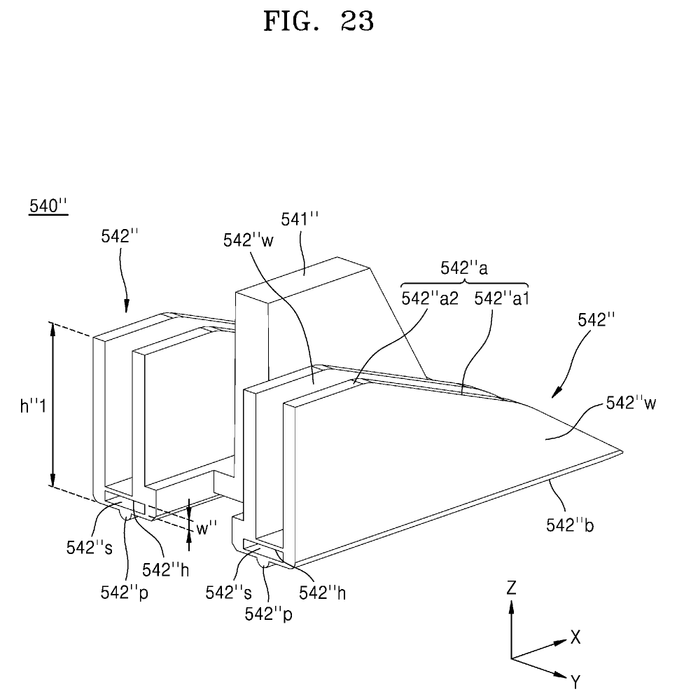

FIG. 21 is a perspective view of the moving member according to the other embodiment of the present disclosure, FIG. 22 is a side view of the moving member of FIG. 21, and FIG. 23 is a perspective view of the moving member of FIG. 21, viewed from another direction.

Referring to FIGS. 21 to 23, a moving member 540’’ according to the other embodiment of the present disclosure may include a body part 541’’ and wedge parts 542’’.

The moving member 540’’ according to the other embodiment of the present disclosure is different from the moving member 540 according to an embodiment of the present disclosure in that there is a difference in a hollow 542’’h, and thus, a detailed description will be provided below with a focus on this difference.

The moving member 540’’ according to the present embodiment may be applied to various cartridges 500. For example, the moving member 540’’ may be applied to a cartridge 500 that includes a cover 510, a housing 520, and a plurality of staples 530, such as the cartridge 500 in the above-described embodiment, and may be applied as is or modified within a permissible range of equivalence, depending on the needs of other cartridges 500 with various structures.

Referring to FIGS. 21 to 23, a first region a1 may be positioned in the wedge part 542’’. At this time, a height of the moving member 540’’ may be adjusted as a bottom surface 542’’b of the wedge part 542’’ is deformed by an external force.

A protrusion 542’’p, which protrudes toward the housing 520 from the bottom surface 542’’b and extends in the first direction, may be formed on the wedge part 542’’ of the moving member 540’’. The protrusion 542’’p may be a region that is in contact with the housing 520 when the moving member 540’’ moves in the first direction.

A length of the protrusion 542’’p in the first direction may be configured to be less than a length of the bottom surface 542’’b in the first direction. In other words, one end of the protrusion 542’’p may be positioned at the same position as an end portion of the moving member 540’’ facing the proximal end 100p of the cartridge 500.

Referring to FIG. 23, the wedge part 542’’ may further include the hollow 542’’h positioned adjacent to the protrusion 542’’p and formed in the first direction.

The hollow 542’’h may be positioned between a pair of wedges 542’’w positioned on one side of the body part 541’’. Further, it may be said that the hollow 542’’h may be positioned on one side (an upper side based on FIG. 23) of a support 542’’s.

That is, the protrusion 542’’p may be formed on the bottom surface 542’’b, which is one surface of the support 542’’s, and the hollow 542’’h may be formed on a surface opposite to the one surface. The hollow 542’’h may have a preset width w’’. When the wedge part 542’’ receives an external force, the support 542’’s may be pressed and deformed in a direction toward the protrusion 542’’p.

In an optional embodiment, a separate elastic member (not shown) may be positioned in the hollow 542’’h. The elastic member may be, for example, a spring, but is not limited thereto, and may be variously modified within the technical concept of being positioned in the hollow and having an elastic restoring force.

One side of the elastic member may be positioned on an upper surface (an upper side based on FIG. 23) of the hollow 542’’h, and another side of the elastic member may be positioned on a lower surface (a lower side based on FIG. 23) of the hollow 542’’h. In other words, the elastic member may support the wedge part 542’’ in a vertical direction (the Z-axis direction) inside the hollow 542’’h.

Accordingly, when an external force is applied to the moving member 540’’, the wedge parts 542’’ may be pressed in a direction toward the first jaw 101, and in this case, a wedge-part maximum height h’’1 may decrease. For example, the wedge-part maximum height h’’1 may be adjusted within the range defined by the width w’’ of the hollow 542’’h.

For example, when the external force applied to the moving member 540’’ is removed, the hollow 542’’h, which was pressed downward, may return to its original shape due to the elastic restoring force of the elastic member, and the wedge-part maximum height h’’1 may also return to its original height.

A length of the hollow 542’’h in the first direction and the length of the protrusion 542’’p in the first direction may be configured to be substantially equal to each other. Accordingly, an end portion of the wedge part 542’’ facing the proximal end 100p, an end portion of the hollow 542’’h facing the proximal end 100p, and an end portion of the protrusion 542’’p facing the proximal end 100p may all be positioned on the same plane.

In the moving member 540’’ according to the other embodiment of the present disclosure, the wedge-part maximum height h’’1 may also be adjusted based on the thickness of the body tissue on which stapling is performed, as shown in FIG. 17, and as a result, formed heights of the staples 530 withdrawn from the cartridge 500 may be adjusted.

In other words, as the thickness of the body tissue increases, the formed height of the staple 530 increases, and as the thickness of the body tissue decreases, the formed height of the staple 530 decreases.

The moving member 540’’ according to the other embodiment of the present disclosure has the same configuration, operating principle, and effect as the moving member 540 according to an embodiment of the present disclosure, except for the hollow 542’’h, and accordingly, a detailed description thereof will be omitted in the overlapping range.

Each of the embodiments described above is an embodiment that can be implemented independently, but it is also possible for the structures of each embodiment to be applied to other embodiments in a combined manner.

As described above, the cartridge according to the embodiments of the present disclosure may have the wedge part having a region deformable by an external force, thereby allowing final formed heights of the staples to be adjusted based on the thickness of the body tissue on which stapling is performed.

In the case of the related art, the final formed heights of the staples were predetermined based on the type of cartridge in which the staples were mounted. As a result, an appropriate cartridge had to be selected during stapling in consideration of the thickness and characteristics of the body tissue.

In case that a cartridge having a final formed height that is too high compared to the thickness of the body tissue is selected, a problem may arise in which the tissue is not sufficiently compressed. Conversely, in case that a cartridge with a final formed height that is too low is selected, the tissue may be excessively compressed and torn, or a stapling instrument may be damaged due to an excessive reactive force from the body tissue.

For example, the cartridge according to an embodiment of the present disclosure employs the moving member with a height that is deformable according to an external force, thereby allowing a single cartridge to respond to various body tissue thicknesses.

Further, even when the thickness of the body tissue varies within a single stapling section, the height of the moving member may deform according to the thickness change, thereby allowing the final formed heights of the staples to be individually adjusted to match the thickness of the body tissue.

According to an embodiment of the present disclosure, during stapling in laparoscopic surgery or various other surgical procedures, a formed height of a staple can be flexibly adjusted within a preset range based on a thickness of the body tissue.

The present disclosure has been described above with a focus on exemplary embodiments. It will be understood by those skilled in the art that various changes in form and details may be made therein without departing from the essential features of the present disclosure. Therefore, the disclosed embodiments should be considered in descriptive sense only and not for purposes of limitation. The scope of the present disclosure is defined not by the detailed description of the disclosure but by the appended claims, and all differences within the scope will be construed as being included in the present disclosure.

What is claimed is:

1. A cartridge of a surgical instrument, which is applied to an end tool of the surgical instrument, the cartridge comprising:
a housing;
a cover covering one surface of the housing and having a slit defined along a first direction, wherein the first direction is a longitudinal direction of the housing;
a plurality of staples disposed inside the housing; and
a moving member disposed inside the housing, configured to move along the slit relative to the housing, and having a deformable region that is configured to bedeformable according to an external force.

2. The cartridge of claim 1, wherein the deformable region of the moving member has a lower rigidity compared to other regions.

3. The cartridge of claim 1, wherein the moving member includes:
a body part disposed in the slit; and
a wedge part disposed on both sides of the body part and configured to push the plurality of staples upward.

4. The cartridge of claim 3, wherein
the deformable region is located on the wedge part, and
the wedge part includes a contact surface configured to contact the plurality of staples and an opposite surface to the contact surface, wherein the contact surface is deformable by the external force.

5. The cartridge of claim 4, wherein the wedge part of the moving member includes a wedge hole positioned at a first predetermined height of the deformable region and extending in the first direction.

6. The cartridge of claim 5, wherein the wedge hole is positioned closer to the contact surface than to the opposite surface.

7. The cartridge of claim 5, wherein
the wedge part includes a first end positioned toward a distal end of the cartridge and a second end positioned toward a proximal end of the cartridge, and
the wedge hole is positioned closer to the second end than to the first end.

8. The cartridge of claim 4, wherein the wedge part of the moving member includes a wedge groove positioned at a second predetermined height of the deformable region and open toward a proximal end of the cartridge.

9. The cartridge of claim 3, wherein
The deformable region is positioned on the wedge part, and
the wedge part includes a contact surface configured to contact the plurality of staples and an opposite surface to the contact surface, wherein the opposite surface is deformable by the external force.

10. The cartridge of claim 9, wherein the wedge part has a protrusion protruding from the opposite surface toward the housing and extending in the first direction.

11. The cartridge of claim 10, wherein the wedge part further includes a hollow positioned adjacent to the protrusion and defined along the first direction.

12. An end tool comprising:
a first jaw configured to accommodate at least one region of an operation member that is movable in a first direction;
a second jaw facing the first jaw and configured to be rotatable around the first jaw; and
a cartridge including a cover disposed in the first jaw and having a slit defined along the first direction, and a plurality of staples configured to be withdrawn toward the second jaw in response to a movement of the operation member,
wherein a height of each of the plurality of staples is adjusted by a force applied when the plurality of staples are withdrawn.

13. The end tool of claim 12, wherein
the cartridge further includes a moving member configured to push the plurality of staples upward in response to the movement of the operation member,
wherein the moving member has a deformable region that is configured to be deformable according to an external force.

14. The end tool of claim 13, wherein the moving member includes:
a body part disposed in the slit; and
a wedge part disposed on each of opposite sides of the body part and configured to push the plurality of staples upward.

15. The end tool of claim 14, wherein
the deformable region is located on the wedge part, and
the wedge part includes a contact surface configured to contact the plurality of staples and an opposite surface to the contact surface, wherein the contact surface is deformable by the external force.

16. The end tool of claim 15, wherein the wedge part of the moving member includes a wedge hole positioned at a first predetermined height of the deformable region and extending in the first direction.

17. The end tool of claim 15, wherein the wedge part of the moving member includes a wedge groove positioned at a second predetermined height of the deformable region and open toward a proximal end of the end tool.

18. The end tool of claim 14, wherein
the deformable region is located on the wedge part, and
the wedge part includes a contact surface configured to contact the plurality of staples and an opposite surface to the contact surface, wherein the opposite surface is deformable by the external force.

19. A surgical instrument comprising:
an end tool including:
a first jaw configured to accommodate at least one region of an operation member that is movable in a first direction,
a second jaw facing the first jaw and configured to be rotatable around the first jaw, and
a cartridge including a cover disposed in the first jaw and having a slit defined along the first direction, and a plurality of staples configured to be withdrawn toward the second jaw in response to a movement of the operation member,
wherein a height of each of the plurality of staples is adjusted by a force applied when the plurality of staples are withdrawn;
a manipulation part configured to control an operation of the end tool; and
a connection part configured to connect the manipulation part to the end tool.

20. The surgical instrument of claim 19, wherein the end tool is configured to perform a yaw rotation around one shaft and a pitch rotation around another shaft different from the one shaft.
